SEQUENCE LISTING (90 sequences)

SEQ ID NO: 1 - DNA, 21 bp, synthetic construct
  source 1..21: mol_type = other DNA
caagtacaacgccaacaagac

SEQ ID NO: 2 - DNA, 19 bp, synthetic construct
  source 1..19: mol_type = other DNA
gtccaggctccatcctttt

SEQ ID NO: 3 - DNA, 19 bp, synthetic construct
  source 1..19: mol_type = other DNA
aatgggcatcctgaagctg

SEQ ID NO: 4 - DNA, 17 bp, synthetic construct
  source 1..17: mol_type = other DNA
catgtggcagtgttgca

SEQ ID NO: 5 - DNA, 19 bp, synthetic construct
  source 1..19: mol_type = other DNA
accatggtgtgtgctggag

SEQ ID NO: 6 - DNA, 18 bp, synthetic construct
  source 1..18: mol_type = other DNA
aacaaagctggtcacacc

SEQ ID NO: 7 - DNA, 20 bp, synthetic construct
  source 1..20: mol_type = other DNA
gggaaaacaaatgatgtggg

SEQ ID NO: 8 - DNA, 24 bp, synthetic construct
  source 1..24: mol_type = other DNA
tttccagacagtgtgacagatacc

SEQ ID NO: 9 - DNA, 21 bp, synthetic construct
  source 1..21: mol_type = other DNA
tcaaggcatatctgacaatcc

SEQ ID NO: 10 - DNA, 20 bp, synthetic construct
  source 1..20: mol_type = other DNA
cagtagctttagccagggca

SEQ ID NO: 11 - DNA, 20 bp, synthetic construct
  source 1..20: mol_type = other DNA
tcctgatcctcctgcttgtc

SEQ ID NO: 12 - DNA, 19 bp, synthetic construct
  source 1..19: mol_type = other DNA
tcatgcagagctcaccgtt

SEQ ID NO: 13 - DNA, 20 bp, synthetic construct
  source 1..20: mol_type = other DNA
atcaacgaacagtgggtggt

SEQ ID NO: 14 - DNA, 20 bp, synthetic construct
  source 1..20: mol_type = other DNA
ctcattcccctccaggactt

SEQ ID NO: 15 - DNA, 20 bp, synthetic construct
  source 1..20: mol_type = other DNA
ttgtgctagctgtgctgctc

SEQ ID NO: 16 - DNA, 19 bp, synthetic construct
  source 1..19: mol_type = other DNA
gcacttgatcattttgcgg

SEQ ID NO: 17 - DNA, 19 bp, synthetic construct
  source 1..19: mol_type = other DNA
attctctcctgtttggcgg

SEQ ID NO: 18 - DNA, 20 bp, synthetic construct
  source 1..20: mol_type = other DNA
tcatggctttgtgggtctct

SEQ ID NO: 19 - DNA, 20 bp, synthetic construct
  source 1..20: mol_type = other DNA
cacagggcacattcaccagt

SEQ ID NO: 20 - DNA, 20 bp, synthetic construct
  source 1..20: mol_type = other DNA
aattcatcgtgacgtttggc

SEQ ID NO: 21 - DNA, 20 bp, synthetic construct
  source 1..20: mol_type = other DNA
gcaagatccagattggcaac

SEQ ID NO: 22 - DNA, 20 bp, synthetic construct
  source 1..20: mol_type = other DNA
cagatggctggacgcttact

SEQ ID NO: 23 - DNA, 20 bp, synthetic construct
  source 1..20: mol_type = other DNA
ctcacagctgctcattgcat

SEQ ID NO: 24 - DNA, 20 bp, synthetic construct
  source 1..20: mol_type = other DNA
aactgggctgtgttttcgtc

SEQ ID NO: 25 - DNA, 23 bp, synthetic construct
  source 1..23: mol_type = other DNA
cctccacatggtgacagagtaca

SEQ ID NO: 26 - DNA, 21 bp, synthetic construct
  source 1..21: mol_type = other DNA
ctcttcatactctggggaggc

SEQ ID NO: 27 - DNA, 18 bp, synthetic construct
  source 1..18: mol_type = other DNA
catctgctccctctacca

SEQ ID NO: 28 - DNA, 21 bp, synthetic construct
  source 1..21: mol_type = other DNA
ctttattccatctctctcggt

SEQ ID NO: 29 - DNA, 21 bp, synthetic construct
  source 1..21: mol_type = other DNA
agaagtatgacaacagcctca

SEQ ID NO: 30 - DNA, 19 bp, synthetic construct
  source 1..19: mol_type = other DNA
ccaaagttgtcatggatga

SEQ ID NO: 31 - DNA, 18 bp, synthetic construct
  source 1..18: mol_type = other DNA
catcaccattggcaatga

SEQ ID NO: 32 - DNA, 19 bp, synthetic construct
  source 1..19: mol_type = other DNA
aaggtagtttcgtggatgc

SEQ ID NO: 33 - DNA, 20 bp, synthetic construct
  source 1..20: mol_type = other DNA
agacattgacctcaccaaga

SEQ ID NO: 34 - DNA, 18 bp, synthetic construct
  source 1..18: mol_type = other DNA
ttgtactggcgtggattc

SEQ ID NO: 35 - DNA, 18 bp, synthetic construct
  source 1..18: mol_type = other DNA
ctctgattaacgcccagc

SEQ ID NO: 36 - DNA, 18 bp, synthetic construct
  source 1..18: mol_type = other DNA
gcggtcggtaaagttgtt

SEQ ID NO: 37 - DNA, 19 bp, synthetic construct
  source 1..19: mol_type = other DNA
gtccaggctccatcctttt

SEQ ID NO: 38 - DNA, 17 bp, synthetic construct
  source 1..17: mol_type = other DNA
catgtggcagtgttgca

SEQ ID NO: 39 - DNA, 18 bp, synthetic construct
  source 1..18: mol_type = other DNA
aacaaagctggtcacacc

SEQ ID NO: 40 - DNA, 24 bp, synthetic construct
  source 1..24: mol_type = other DNA
tttccagacagtgtgacagatacc

SEQ ID NO: 41 - DNA, 20 bp, synthetic construct
  source 1..20: mol_type = other DNA
cagtagctttagccagggca

SEQ ID NO: 42 - DNA, 19 bp, synthetic construct
  source 1..19: mol_type = other DNA
tcatgcagagctcaccgtt

SEQ ID NO: 43 - DNA, 19 bp, synthetic construct
  source 1..19: mol_type = other DNA
cacgcgggcgttgattact

SEQ ID NO: 44 - DNA, 20 bp, synthetic construct
  source 1..20: mol_type = other DNA
gtagcagccgtagttgttgt

SEQ ID NO: 45 - DNA, 20 bp, synthetic construct
  source 1..20: mol_type = other DNA
actgggattgagttgcagga

SEQ ID NO: 46 - DNA, 20 bp, synthetic construct
  source 1..20: mol_type = other DNA
aattcatcgtgacgtttggc

SEQ ID NO: 47 - DNA, 20 bp, synthetic construct
  source 1..20: mol_type = other DNA
cagatggctggacgcttact

SEQ ID NO: 48 - DNA, 20 bp, synthetic construct
  source 1..20: mol_type = other DNA
catgttgaagccagggtgtg

SEQ ID NO: 49 - DNA, 21 bp, synthetic construct
  source 1..21: mol_type = other DNA
cttggctgccttctggtatct

SEQ ID NO: 50 - DNA, 21 bp, synthetic construct
  source 1..21: mol_type = other DNA
ctttattccatctctctcggt

SEQ ID NO: 51 - DNA, 19 bp, synthetic construct
  source 1..19: mol_type = other DNA
catgagtccttccacgata

SEQ ID NO: 52 - DNA, 20 bp, synthetic construct
  source 1..20: mol_type = other DNA
tggagttgaaggtagtttcg

SEQ ID NO: 53 - DNA, 20 bp, synthetic construct
  source 1..20: mol_type = other DNA
aagaaccagtctgggatctt

SEQ ID NO: 54 - DNA, 18 bp, synthetic construct
  source 1..18: mol_type = other DNA
gcggtcggtaaagttgtt

SEQ ID NO: 55 - DNA, 29 bp, synthetic construct
  source 1..29: mol_type = other DNA
  misc_feature 22: note = RNA
  misc_feature join(1..21,23..29): note = DNA
  misc_feature 29: note = a blocking group
caagtacaacgccaacaagacnccctggv

SEQ ID NO: 56 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
gtccaggctccatccttttnggcagtv

SEQ ID NO: 57 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
aatgggcatcctgaagctgncaagttv

SEQ ID NO: 58 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
catgtggcagtgttgcatttnccgctav

SEQ ID NO: 59 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
accatggtgtgtgctggagnggtaccv

SEQ ID NO: 60 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
aacaaagctggtcacaccgtnggaccav

SEQ ID NO: 61 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
gggaaaacaaatgatgtgggnccagagv

SEQ ID NO: 62 - DNA, 32 bp, synthetic construct
  source 1..32: mol_type = other DNA
  misc_feature join(1..24,26..32): note = DNA
  misc_feature 25: note = RNA
  misc_feature 32: note = a blocking group
tttccagacagtgtgacagataccnttatagv

SEQ ID NO: 63 - DNA, 30 bp, synthetic construct
  source 1..30: mol_type = other DNA
  misc_feature join(1..22,24..30): note = DNA
  misc_feature 23: note = RNA
  misc_feature 30: note = a blocking group
tcaaggcatatctgacaatccanctcgttv

SEQ ID NO: 64 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
cagtagctttagccagggcanttcaagv

SEQ ID NO: 65 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
tcctgatcctcctgcttgtcngcccttv

SEQ ID NO: 66 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
tcatgcagagctcaccgttnctcctcv

SEQ ID NO: 67 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
tgtggtggctccctcatcaancgaactv

SEQ ID NO: 68 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
cacgcgggcgttgattactngcacgav

SEQ ID NO: 69 - DNA, 30 bp, synthetic construct
  source 1..30: mol_type = other DNA
  misc_feature join(1..22,24..30): note = DNA
  misc_feature 23: note = RNA
  misc_feature 30: note = a blocking group
gaaactccttgtgctagctgtgnctgctgv

SEQ ID NO: 70 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
gtagcagccgtagttgttgtnattcctv

SEQ ID NO: 71 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
  modified_base 22: mod_base = OTHER; note = uracil
ggctcatgccattgaccctcnttaatgv

SEQ ID NO: 72 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
  modified_base 22: mod_base = OTHER; note = uracil
actgggattgagttgcaggantcatgcv

SEQ ID NO: 73 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
cacagggcacattcaccagtngactagv

SEQ ID NO: 74 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
aattcatcgtgacgtttggcnaatgtav

SEQ ID NO: 75 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
gcaagatccagattggcaacnacctagv

SEQ ID NO: 76 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
cagatggctggacgcttactngccccgv

SEQ ID NO: 77 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
tttccagtgtgggggcatcnctggtcv

SEQ ID NO: 78 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
catgttgaagccagggtgtgnggaaggv

SEQ ID NO: 79 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
  modified_base 21: mod_base = OTHER; note = uracil
tgccaccaaattgagccgtnttcatgv

SEQ ID NO: 80 - DNA, 29 bp, synthetic construct
  source 1..29: mol_type = other DNA
  misc_feature join(1..21,23..29): note = DNA
  misc_feature 22: note = RNA
  misc_feature 29: note = a blocking group
cttggctgccttctggtatctngtgcaav

SEQ ID NO: 81 - DNA, 26 bp, synthetic construct
  source 1..26: mol_type = other DNA
  misc_feature join(1..18,20..26): note = DNA
  misc_feature 19: note = RNA
  misc_feature 26: note = a blocking group
catctgctccctctaccangctgggv

SEQ ID NO: 82 - DNA, 29 bp, synthetic construct
  source 1..29: mol_type = other DNA
  misc_feature join(1..21,23..29): note = DNA
  misc_feature 22: note = RNA
  misc_feature 29: note = a blocking group
ctttattccatctctctcggtngcaggtv

SEQ ID NO: 83 - DNA, 26 bp, synthetic construct
  source 1..26: mol_type = other DNA
  misc_feature join(1..18,20..26): note = DNA
  misc_feature 19: note = RNA
  misc_feature 26: note = a blocking group
atgttcgtcatgggtgtgnaaccagv

SEQ ID NO: 84 - DNA, 27 bp, synthetic construct
  source 1..27: mol_type = other DNA
  misc_feature join(1..19,21..27): note = DNA
  misc_feature 20: note = RNA
  misc_feature 27: note = a blocking group
catgagtccttccacgatanccaaacv

SEQ ID NO: 85 - DNA, 30 bp, synthetic construct
  source 1..30: mol_type = other DNA
  misc_feature join(1..22,24,30): note = DNA
  misc_feature 23: note = RNA
  misc_feature 30: note = a blocking group
agaagagctacgagctgcctgancggcccv

SEQ ID NO: 86 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
  modified_base 22: mod_base = OTHER; note = uracil
tggagttgaaggtagtttcgntggattv

SEQ ID NO: 87 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
tatgctcatgtggtgttgagngaaaggv

SEQ ID NO: 88 - DNA, 28 bp, synthetic construct
  source 1..28: mol_type = other DNA
  misc_feature join(1..20,22..28): note = DNA
  misc_feature 21: note = RNA
  misc_feature 28: note = a blocking group
aagaaccagtctgggatcttngtactcv

SEQ ID NO: 89 - DNA, 26 bp, synthetic construct
  source 1..26: mol_type = other DNA
  misc_feature join(1..18,20..26): note = DNA
  misc_feature 19: note = RNA
  misc_feature 26: note = a blocking group
ctctgattaacgcccagcngttttgv

SEQ ID NO: 90 - DNA, 26 bp, synthetic construct
  source 1..26: mol_type = other DNA
  misc_feature join(1..18,20..26): note = DNA
  misc_feature 19: note = RNA
  misc_feature 26: note = a blocking group
gcggtcggtaaagttgttnccattav

(19) United States
(12) Patent Application Publication
(10) Pub. No.: US 2024/0336969 A1
(43) Pub. Date: Oct. 10, 2024

(54) A METHOD OF MONITORING THE HEALTH OF A SUBJECT

(71) Applicants: Agency for Science, Technology and Research, Singapore (SG); National University of Singapore, Singapore (SG)

(72) Inventors: Lian Chye Winston Koh, Singapore (SG); Chin Meng Khoo, Singapore (SG); E Shyong Tai, Singapore (SG)

(21) Appl. No.: 18/294,675

(22) PCT Filed: Aug. 5, 2022
(86) PCT No.: PCT/SG2022/050563

§ 371 Date: Feb. 2, 2024

(30) Foreign Application Priority Data
Aug. 5, 2021 (SG) 10202108592Y
Sep. 24, 2021 (SG) 10202110630Y

(51) Int. Cl. C12Q 1/6883

(52) U.S. Cl. CPC C12Q 1/6883; C12Q 2600/158

(57) ABSTRACT

This disclosure relates to a method of assessing/monitoring the health of a subject, the method comprising subjecting a pancreas-associated polynucleotide to one or more amplification step in the presence of a mixture comprising a control nuclei acid, a surfactant and an oligonucleotide primer and/or probe capable of hybridizing with the target nucleic acid, wherein the oligonucleotide primer and/or probe comprises a cleavage site and a cleavable 3′ end. In one embodiment, the pancreas-associated polynucleotide is cell-free RNA and the method comprises annealing the target nucleic acid and performing a reverse transcription prior to the one or more amplification step. Also disclosed are primer and/or primer sets as disclosed herein and kits for use thereof.

### RELATED APPLICATIONS

This application is the U.S. National Stage of International Application No. PCT/SG2022/050563, filed Aug. 5, 2022, which designates the U.S., published in English, and claims priority under 35 U.S.C. § 119 or 365(c) to Singapore Application No. 10202108592Y, filed Aug. 5, 2021 and Singapore Application No. 10202110630Y, filed Sep. 24, 2021. The entire teachings of the above applications are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:
a) File name: 5975.1025-001 Corrected Sequence Listing.xml; created Apr. 29, 2024, 123,062 Bytes in size.

### TECHNICAL FIELD

The present disclosure relates broadly to a method of monitoring the health of a subject. In particular, the present disclosure relates to the determining, detecting and/or quantifying a pancreas-associated nucleic acid in a subject.

### BACKGROUND

Bariatric surgery results in sustained weight loss and improvements in glucose homeostasis. However, the lack of accessible non-invasive tools to examine molecular alterations occurring in the pancreas limits the understanding of the causes and recovery of glucose homeostasis.

After bariatric surgery, patients exhibit drastic improvements in metabolic health as reflected in their loss of body weight/BMI, and reversal from diabetes mellitus. The reversal in diabetes mellitus suggests an improvement to pancreatic beta-cell function/health on the background of improvement to insulin sensitivity. However, changes in molecular function of pancreatic islets that resulted in these improvements remain an area of great uncertainty.

Recent single cell RNA sequencing studies using donor pancreases obtained from deceased individuals of different BMI and obesity levels, have revealed transcriptional changes within α- and β-cell types that directly correlate to BMI. When taken in consideration the significant reduction in BMI following bariatric surgery, reversal in gene expression can be expected from these pancreatic cell types. Unfortunately, it is almost impossible to access pancreatic tissues/cells from living patients to study the cellular changes with weight loss, even less so when considering the study to examine the trajectories of cellular changes across multiple time points following surgery.

Therefore, there is a need to provide an alternative method of monitoring the health of a subject. In particular, there is a need to provide an alternative method of determining, detecting, and/or quantifying a pancreas-associated nucleic acid in a subject

### SUMMARY

In one aspect, the present invention provides a method for assessing/monitoring the health of a subject, the method comprising determining, detecting or quantifying a pancreas-associated polynucleotide in a sample from the subject.

In some examples, wherein the pancreas-associated polynucleotide comprises a pancreas-associated RNA, optionally the RNA comprises a cell-free and/or circulating RNA.

In some examples, wherein the RNA comprises a messenger RNA (mRNA) or part thereof.

In some examples, wherein the method is performed before, during and/or after the subject is being administered a treatment regimen, optionally the method is performed pre and/or post-bariatric surgery.

In some examples, wherein the method comprising annealing the target nucleic acid in the presence of a control nucleic acid, and subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

In some examples, wherein the amplification step of the nucleic acid is performed in the presence of three parts surfactant to one part amplification mixture, optionally the method comprises two amplification steps.

In some examples, wherein the method further comprises a step of freeze and thawing the amplified mixture.

In some examples, wherein the method further comprises performing one or more of the following: RNaseH2-dependent PCR, emulsion PCR, and/or CoT PCR.

In some examples, wherein determining, detecting or quantifying the pancreas-associated polynucleotide comprises performing quantitative polymerase chain reaction (qPCR).

In some examples, wherein the method further comprises reverse transcribing the pancreas-associated RNA.

In some examples, wherein the method further comprises subjecting the products obtained from the preceding step (e.g., the cDNA obtained after reverse transcribing the pancreas-associated RNA) to RNaseH2-dependent polymerase chain reaction (rhPCR).

In some examples, wherein the method further comprises subjecting the products obtained from the preceding step to CoT PCR.

In some examples, wherein the method further comprises adding a control nucleic acid to the sample, optionally wherein the control nucleic acid is added to the sample at a constant amount to thereby normalizes of the amplification efficiency across a plurality of samples, optionally the control nucleic acid is added to the sample at about 10² to 10¹⁰ copies.

In some examples, wherein the sample comprises a blood sample, optionally the blood sample is a plasma sample.

In some examples, wherein the pancreas-associated polynucleotide comprises the mRNA of a pancreatic-specific gene or part thereof.

In some examples, wherein the pancreatic-specific gene is at least one selected from the group consisting of KLK1, CTRB1, ERP27, IAPP, PRSS1, CELA3A, PLA2G1B, PNLIP, CUZD1, CPB1, CPA1, CLPS, INS and GCG.

In some examples, wherein the pancreatic-specific gene comprises GCG.

In some examples, wherein the subject has, is suspected to have, or is diagnosed to have a metabolic disease.

In another aspect, the present invention provides a primer or a primer set comprising one or more primers having a sequence listed in Table 1, Table 2, Table 3 and/or Table 4, or a part thereof, or a sequence sharing at least about 70% sequence identity thereto.

In yet another aspect, the present invention provides a kit for assessing/monitoring the health of a subject, the kit comprising one or more of the following: one or more primers/primer set primers having a sequence listed in Table 1, Table 2, Table 3 and/or Table 4, or a part thereof, or a sequence sharing at least about 70% sequence identity thereto, a control polynucleotide; an rhPCR primer; and a surfactant.

### Definitions

As used herein, the term “biological sample” refers to a sample obtained from a biological subject, including a sample of biological tissue or fluid origin obtained in vivo or in vitro. Hence, a “biological sample” may be a solid biological sample or a liquid biological sample. Examples of a “solid biological sample” may include biopsies, such as an organ biopsy, a tumour biopsy, stools, cell culture, food, plant extracts, and the like. Examples of a “fluid biological sample” or “liquid biological sample” include blood, serum, plasma, sputum, lavage fluid (for example peritoneal lavage), cerebrospinal fluid, urine, vaginal discharge, semen, sweat, tears, saliva, and the like. As used herein, the terms “blood”, “plasma”, and “serum” encompass fractions or processed portions thereof. Similarly, where a sample is taken from a biopsy, swab, smear, etc., the “sample encompass a processed fraction or portion derived from the biopsy, swab, smear, etc.

As used herein, the term “detecting” includes the step of determining the presence and/or absence of cfRNA. In some examples, the term “detecting” may further include the step of quantification of the cfRNA detected in the sample.

As used herein, the term “isolated” refers to a nucleic acid that is removed from its natural environment. An “isolated” nucleic acid is typically partially purified.

As used herein, the term “nucleic acid” or “polynucleotide” refers to a nucleotide sequence that typically includes nucleotides comprising an A, G, C, T or U base. In some examples, nucleotide sequences may include other bases such as inosine, methylcytosine, hydroxymethylcytosine, methylinosine, methyladenosie and/or thiouridine, and the like. The term “nucleic acid” or “polynucleotide” may include both single and/or double stranded deoxyribonucleic acid (DNA) and ribonucleic acid (RNA), including environmental DNA (eDNA), genomic DNA, bacterial DNA, viral DNA, cell-free DNA (cfDNA), complementary RNA (cRNA), messenger RNA (mRNA), transfer RNA (TRNA), microRNA (miRNA), circulating cell free RNA/cell free RNA (cfRNA), extracellular RNA, circulating tumour RNA (ctRNA), bacterial RNA, viral RNA, ribosomal RNA (rRNA) and the like.

As used in herein, the term “target nucleic acid” or “target polynucleotide” refers to nucleic acid whose presence is to be detected or measured or whose function, interactions or properties are to be studied. Therefore, a target nucleic acid includes essentially any nucleic acid for which a detectable probe (e.g., oligonucleotide probe) or assay exists, or can be produced or isolated by one skilled in the art. Target nucleic acid may include disease markers, viral DNA and/or RNA, bacterial DNA and/or RNA, tumour markers, and the like.

As used herein, the term “real time” refers to the actual time during which a process or event occurs and/or tracking of temporal changes and/or trajectories of cellular changes in samples drawn from different time points.

As used herein, the term “surfactant” refers to a composition that stabilizes water-in-oil droplets that is capable of or that can encapsulate nucleic acids (such as DNA, cDNA, cfDNA, RNA, cfRNA, and the like). In some examples, the surfactant may comprise a particular repeat unit comprising a perfluoropolyether and a polyalkylene oxide unit. In some examples, the surfactant may be one or more of fluorosurfactant, non-ionic surfactant, anionic surfactant, cationic surfactant, amphoteric surfactant, and the like. In some examples, the fluorosurfactant may be synthesized by coupling oligomeric perfluorinated polyethers (PFPE) with polyethyleneglycol (PEG).

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” or “attached” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “adjacent” used herein when referring to two elements refers to one element being in close proximity to another element and may be but is not limited to the elements contacting each other or may further include the elements being separated by one or more further elements disposed therebetween. For example, the cleavage compound as described herein cleaves the oligonucleotide (e.g. primer, probe, and the like) within or adjacent to the cleavage domain. Thus, the term “adjacent” means that the cleavage compound cleaves the oligonucleotide at either the 5′-end or the 3′ end of the cleavage domain. In some examples of the present disclosure, the cleavage reactions yield a 5′-phosphate group and a 3′-OH group.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. As used herein, the term “substantially no” or “very low” refers to a sequence homology of less than at least 20%, or 19%, or 18%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.9%, or 0.8%, or 0.7%, or 0.6%, or 0.5%, or 0.4%, or 0.3%, or 0.2%, or 0.1%, or 0.01% sequence homology to the target nucleic acid (for example any human gene). In some examples, the term “substantially no” or “very low” sequence homology refers to the control gene having substantially different sequence to the target nucleic acid (for example any human gene). In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of methods of assessing/monitoring the health (e.g. a pancreatic health or a metabolic health) of a subject are disclosed herein after. Also disclosed are methods of amplifying and/or quantifying a nucleic acid are disclosed hereinafter. Also disclosed are methods of detecting and/or determining the presence and/or the amount of a target nucleic acid (e.g., a pancreatic-specific nucleic acid).

In one aspect, there is provided a method for assessing/monitoring the health (e.g., a pancreatic health or a metabolic health) of a subject, the method comprising determining, detecting or quantifying a pancreatic-specific nucleic acid in a sample from the subject.

As used herein, the term “pancreatic-specific nucleic acid” refers to nucleic acid that are highly expressed have four or more folds-change expression in the pancreas as compared to in other tissues. In some examples, the pancreatic-specific nucleic acid is biologically significant/relevant, and/or highly expressed (such as more than 2 to 10 times fold-change when compared to other tissues), and/or falls into the category of “tissue-enriched genes” in pancreatic tissues. In some examples, the pancreatic-specific nucleic acid is biologically significant and/or highly expressed according to databases known in the art (such as Human Protein Atlas).

In some examples, the pancreatic-specific nucleic acid are genes that are highly expressed and/or have four or more folds-change expression in the pancreas as compared to in other tissues.

A pancreatic-specific gene may be a gene that is expressed at a higher level (e.g., at least about 2 times, about 3 times, about 4 times or about 5 times higher) in pancreas relative to the baseline expression across all tissues in a subject e.g., a human subject.

In some examples, the expression is 2, 3, 4, 5, 6, 7, 8, 9, 10, or more folds-change higher than mRNA levels in a group of tissues compared to other tissues in the databases known in the art (such as Human Protein Atlas). In some examples, highly expressed pancreatic-specific nucleic acid when compared to other tissues may include, but is not limited to, 2 times fold-change, 3 times fold-change, 4 times fold-change, 5 times fold-change, 6 times fold-change, 7 times fold-change, 8 times fold-change, 9 times fold-change, or 10 times fold-change, and the like. In some examples, the pancreatic-specific nucleic acid expression is 4 times fold-change higher than mRNA levels in a group of tissues compared to other tissues in the database known in the art.

In some examples, the group of tissues compared to other tissues in the database known in the art (such as Human Protein Atlas) may comprise, but is not limited to, 1 to 2 tissues, 1 to 3 tissues, 1 to 4 tissues, 1 to 5 tissues, 1 to 6 tissues, 1 to 7 tissues, 1 to 8 tissues, 1 to 9 tissues, 1 to 10 tissues, 2 to 3 tissues, 2 to 4 tissues, 2 to 5 tissues, 2 to 6 tissues, 2 to 7 tissues, 2 to 8 tissues, 2 to 9 tissues, 2 to 10 tissues, 3 to 4 tissues, 3 to 5 tissues, 3 to 6 tissues, 3 to 7 tissues, 3 to 8 tissues, 3 to 9 tissues, 3 to 10 tissues, 4 to 5 tissues, 4 to 6 tissues, 4 to 7 tissues, 4 to 8 tissues, 4 to 9 tissues, 4 to 10 tissues, 5 to 6 tissues, 5 to 7 tissues, 5 to 8 tissues, 5 to 9 tissues, 5 to 10 tissues, 6 to 7 tissues, 6 to 8 tissues, 6 to 9 tissues, 6 to 10 tissues, 7 to 8 tissues, 7 to 9 tissues, 7 to 10 tissues, 8 to 9 tissues, 8 to 10 tissues, 9 to 10 tissues, and the like. In some examples, the group of tissues compared to other tissues in the database known in the art is 2 to 5 tissues.

In some examples, the pancreas-associated nucleic acid comprises a pancreas-associated RNA. In some examples, the RNA comprises a cell-free RNA (cfmRNA). In some examples, the RNA comprises a messenger RNA (mRNA) or part thereof. In some examples, the RNA does not comprise microRNA (miRNA). In some examples, the pancreas-associated nucleic acid comprises the mRNA of a pancreatic-specific gene or part thereof.

In various embodiments, the techniques are specifically tailored for cell-free circulating mRNA which are longer in size compared to miRNA. miRNA is biologically a different class of RNA compared to tissue specific cell-free mRNA (cfmRNA). They have different biochemical properties. miRNA is smaller in size and requires different quantification techniques as compared to cfmRNA.

In some examples, the method comprises detecting and/or determining the presence of 2 or more, 3 or more, 4 or more, 5 or more, 6 or more, 7 or more, 8 or more, 9 or more, 10 or more, 11 or more, 12 or more, 13 or more, or 14 or more pancreatic-specific nucleic acids.

In various embodiments, the method comprises determining, detecting and/or quantifying at least about 2, at least about 3, at least about 4, at least about 5, at least about 6, at least about 7, at least about 8, at least about 9, at least about 10, at least about 11, at least about 12, at least about 13 or at least about 14 pancreatic-specific nucleic acids.

In various embodiments, the method comprises determining, detecting and/or quantifying the RNA/mRNA of at least about 2, at least about 3, at least about 4, at least about 5, at least about 6, at least about 7, at least about 8, at least about 9, at least about 10, at least about 11, at least about 12, at least about 13 or at least about 14 pancreatic-specific genes selected from the group consisting of Kallikrein 1 (KLK1), Chymotrypsinogen B1 (CTRB1), Endoplasmic Reticulum Protein 27 (ERP27), Islet Amyloid Polypeptide (IAPP), Serine Protease 1 (PRSS1), Chymotrypsin like Elastase 3A (CELA3A), Phospholipase A2 Group IB (PLA2G1B), Pancreatic triacylglycerol lipase (PNLIP), CUB and Zona Pellucida Like Domains 1 (CUZD1), Carboxypeptidase B1 (CPB1), Carboxypeptidase A1 (CPA1), Colipase (CLPS), insulin (INS), and Glucagon (GCG), or part thereof.

In some examples, the pancreatic-specific nucleic acid identified includes genes such as, but is not limited to, Kallikrein 1 (KLK1), Chymotrypsinogen B1 (CTRB1), Endoplasmic Reticulum Protein 27 (ERP27), Islet Amyloid Polypeptide (IAPP), Serine Protease 1 (PRSS1), Chymotrypsin like Elastase 3A (CELA3A), Phospholipase A2 Group IB (PLA2G1B), Pancreatic triacylglycerol lipase (PNLIP), CUB and Zona Pellucida Like Domains 1 (CUZD1), Carboxypeptidase B1 (CPB1), Carboxypeptidase A1 (CPA1), Colipase (CLPS), insulin (INS), or Glucagon (GCG), and the like. In some examples, the pancreatic-specific gene is at least one selected from the group consisting of KLK1, CTRB1, ERP27, IAPP, PRSS1, CELA3A, PLA2G1B, PNLIP, CUZD1, CPB1, CPA1, CLPS, INS and GCG.

In some examples, the one or more pancreatic-specific nucleic acid comprises Kallikrein 1 (KLK1), Chymotrypsinogen B1 (CTRB1), Endoplasmic Reticulum Protein 27 (ERP27), Islet Amyloid Polypeptide (IAPP), Serine Protease 1 (PRSS1), Chymotrypsin like Elastase 3A (CELA3A), Phospholipase A2 Group IB (PLA2G1B), Pancreatic triacylglycerol lipase (PNLIP), CUB and Zona Pellucida Like Domains 1 (CUZD1), Carboxypeptidase B1 (CPB1), Carboxypeptidase A1 (CPA1), Colipase (CLPS), insulin (INS), and Glucagon (GCG).

In some examples, the pancreatic-specific gene comprises GCG.

In one example, the GCG cfmRNA levels correlated significantly with common metrics of improvement following bariatric surgery such as: haemoglobin A1c levels and percentage of excess weight loss. Hence, the GCG cfmRNA levels may serve as a reliable proxy marker for evaluating the health of a subject or for evaluating tissue specific molecular alterations before and/or after bariatric surgery.

In various embodiments, the pancreatic-specific nucleic acid correlates with one or more clinical biomarkers/measurements or metabolic/surgical outcomes. The correlation may be a positive correlation or a negative correlation. In one example, the GCG cfmRNA levels correlated positively with percentage excess weight loss (% EWL) in a subject. In one example, the GCG cfmRNA levels exhibited an inverse correlation with glycated haemoglobin (HbA1c) levels in a subject.

Embodiments of the method may be used to track temporal changes in the health of a subject before, during and/or after a treatment regimen. The treatment regimen may be a treatment regimen for a metabolic disease such as, but not limited to, obesity and diabetes. The treatment regimen may comprise a surgical procedure, a non-surgical procedure, or both. For example, embodiments of the method may be used to capture temporal changes in the pancreatic health and/or glucose homeostasis in a subject before and/or after a bariatric surgery. Examples of a bariatric surgery include, but are not limited to, gastric bypass surgery, sleeve gastrectomy, gastric band surgery, biliopancreatic diversion with duodenal switch and the like. In various embodiments, the method may also be used to track/monitor/assess the outcome of treatment regimen such as a bariatric surgery. In various embodiments, the method comprises a prognostic method.

In some examples, the subject has, is suspected to have, or is diagnosed to have a metabolic disease.

In some embodiments, the subject has undergone a treatment regimen, e.g., bariatric surgery, for the metabolic disease. In some embodiments, the subject is undergoing a treatment regimen for the metabolic disease. In some embodiments, the subject is recommended for a treatment regimen, e.g., bariatric surgery, for the metabolic disease.

In some examples, the method is performed before, during and/or after the subject is being administered a treatment regimen.

In various embodiments, the sample is collected from the subject before, during and/or after the treatment regimen.

In some examples, the method is performed pre and/or post-bariatric surgery.

In various embodiments, the method comprises obtaining the sample from the subject prior to the determining/detecting/quantifying step. Embodiments of the method may be in vivo, in vitro or ex vivo. In various embodiments, the sample comprises a biological sample. In various embodiments, the sample comprises a liquid/fluid sample. The liquid/fluid sample may include but is not limited to blood, plasma, serum and combinations thereof, and the like. In some examples, the sample comprises a blood sample. In some examples, the blood sample comprises a plasma sample.

In some examples, the one or more pancreatic-specific nucleic acid is obtained from a biological sample. In some examples, the one or more pancreatic-specific nucleic acid is obtained from plasma. In some examples, the one or more pancreatic-specific nucleic acid is a cell free nucleic acid, optionally a circulating cell free nucleic acid.

In various embodiments, the sample comprises cell-free RNA. In various embodiments, the sample comprises circulating cell-free RNA. In various embodiments, the sample comprises circulating cell-free mRNA (cfmRNA). In various embodiments, the RNA is more than about 25 nucleotides (nt), more than about 50 nt or more than about 100 nt in length. In various embodiments, the RNA is at least about 250 nt, at least about 500 nt, at least about 750 nt, at least about 1000 nt, at least about 1250 nt, at least about 1500 nt, at least about 1750 nt, at least about 2000 nt, at least about 2250 nt, at least about 2500 nt, at least about 2750 nt, at least about 3000 nt, at least about 3250 nt, at least about 3500 nt, at least about 3750 nt, at least about 4000 nt, at least about 4250 nt, at least about 4500 nt, at least about 4750 nt, at least about 5000 nt or longer in length.

Embodiments of the method may be a liquid biopsy method. Liquid biopsies, that use circulating mRNA in the plasma, have recently been demonstrated as alternative methods of non-invasive examination of molecular alterations in hard-to-reach tissues like the brain. The inventors of the present disclosure hypothesize that liquid biopsy that specifically target circulating pancreatic mRNA transcripts improvements could be employed to capture changes in pancreatic health in patients undergoing bariatric surgery.

In some embodiments, the step of obtaining the sample from the subject is a non-surgical step, a non-invasive step or a minimally invasive step. In some embodiments, the step of obtaining the sample from the subject comprises withdrawing a blood sample from the subject. In some embodiments, the method further comprises a step of removing particulate blood components from the blood sample to leave behind blood plasma for use in the determining/detecting/quantifying step (for example, through one or more stages of centrifugation to separate the sample into different fractions). In some embodiments, the particulate blood components are selected from the group consisting red blood cells, white blood cells, platelets and combinations thereof. The method may also further comprise one or more further steps of sample processing, such as, but not limited to, extraction of polynucleotide (e.g., RNA), disintegration and/or dissolving of sample or components thereof (e.g., in a solvent), purification and/or sample concentration.

In some examples, the method comprises collecting a sample (such as blood) by spinning the sample twice (such as centrifugation at 1600×g/2000×g and 16000×g) and collecting the resulting sample (such as plasma) within a day after collection. In some examples, the resulting sample (such as plasma) is stored at freezing temperatures (such as −80° C.) before downstream nucleic acid (such as RNA) extraction. In some examples, the method comprises processing the resulting sample (such as plasma from blood) within about 1, 2, 3, 4, 5, or 6, 7, 8, 9, 10, 11, 12 hours. In some examples, the resulting sample is processed within 6 hours.

In various embodiments, the method requires no more than about 10 ml, no more than about 9.5 ml, no more than about 9 ml, no more than about 8.5 ml, no more than about 8 ml, no more than about 7.5 ml, no more than about 7 ml, no more than about 6.5 ml, no more than about 6 ml, no more than about 5.5 ml, no more than about 5 ml, no more than about 4.5 ml, no more than about 4 ml, no more than about 3.5 ml, no more than about 3 ml, no more than about 2.5 ml, no more than about 2 ml, no more than about 1.5 ml, no more than about 1 ml or no more than about 0.5 ml of sample, for example, a sample in the form of plasma.

In various embodiments, the sample is collected from the subject pre-surgery, short-term post-surgery and/or long-term post-surgery.

For example, where the sample is collected from the subject pre-treatment/pre-surgery, the sample may be collected from the subject after the subject is being recommended for the treatment/surgery and before the treatment/surgery takes place. For example, where the sample is collected from the subject pre-treatment/pre-surgery, the sample may be collected from the subject no more than about 6 months, no more than about 5 months, no more than about 4 months, no more than about 3 months, no more than about 2 months, no more than about 1 month, no more than about 3 weeks, no more than about 2 weeks, no more than about 1 week, no more than about 5 days, no more than about 3 days or no more than about 1 day before the treatment regimen/surgery.

For example, where the sample is collected from the subject short-term post-treatment/post-surgery, the sample may be collected at least about 2 weeks, or less than about 3 months after treatment/surgery. For example, the sample may be collected about 2 weeks, about 3 weeks, about 4 weeks, about 5 weeks, about 6 weeks, about 7 weeks, about 8 weeks, about 9 weeks, about 10 weeks or about 11 weeks after treatment/surgery.

For example, where the sample is collected from the subject long-term post-treatment/post-surgery, the sample may be collected at least about 3 months after treatment/surgery. For example, the sample may be collected at least about 3 months, at least about 3.5 months, at least about 4 months, at least about 4.5 months, at least about 5 months, at least about 5.5 months, at least about 6 months, at least about 6.5 months, at least about 7 months, at least about 7.5 months, at least about 8 months, at least about 8.5 months, at least about 9 months, at least about 9.5 months, at least about 10 months, at least about 10.5 months, at least about 11 months, at least about 11.5 months or at least about 12 months after treatment/surgery.

In some examples, the method comprises tracking temporal/real-time changes in a sample. This assay is used to track temporal changes/multiple time points including pre and post bariatric surgery in a cohort of 58 plasma samples drawn from 10 patients. For tracking temporal changes, the sample may be collected from the subject at multiple time points (e.g., at least about 3, at least about 4 or at least about 5 different time points), for example, at pre-treatment/pre-surgery, short-term post-treatment/post-surgery and long-term post/treatment/post-surgery. In some examples, the method comprises collecting a sample from at least 2, 3, 4, 5, 6, 7, 8, 9, 10 time points, or more. In some examples, the method comprises collecting a sample from at least 5 time points. For example, the 5 time points may include but is not limited to, pre-surgery, short-term post-surgery (such as 2 weeks to 1 month), or long-term post-surgery (such as 3 months to 12 months), and the like.

In some examples, the short-term post-surgery is about 1 week to 2 months. In some examples, the short-term post-surgery may be about 1 week to 2 weeks, or about 1 week to 1 month, or about 1 week to 1.5 months, or about 1 week to 2 months, or about 2 weeks to 1 month, or about 2 weeks to 1.5 month, or about 2 weeks to 2 months, or about 3 weeks to 1 month, or about 3 weeks to 1.5 months, or about 3 weeks to 2 months, or about 1 month to 2 months, or about 1.5 months to 2 months, and the like. In some examples, the short-term post-surgery is about 2 weeks to 1 month.

In some examples, the long-term post-surgery is about 3 months to 15 months. In some examples, the long-term post-surgery may be about 3 months to 4 months, or about 3 months to 5 months or about 3 months to 6 months, or about 3 months to 7 months, or about 3 months to 8 months, or about 3 months to 9 months, or about 3 months to 10 months, or about 3 months to 11 months, or about 3 months to 12 months, or about 3 months to 13 months, or about 3 months to 14 months, or about 3 months to 15 months, or about 4 months to 5 months, or about 4 months to 6 months, or about 4 months to 7 months, or about 4 months to 8 months, or about 4 months to 9 months, or about 4 months to 10 months, or about 4 months to 11 months, or about 4 months to 12 months, or about 4 months to 13 months, or about 4 months to 14 months, or about 4 months to 15 months, or about 5 months to 6 months, or about 5 months to 7 months, or about 5 months to 8 months, or about 5 months to 9 months, or about 5 months to 10 months, or about 5 months to 11 months, or about 5 months to 12 months, or about 5 months to 13 months, or about 5 months to 14 months, or about 5 months to 15 months, or about 6 months to 7 months, or about 6 months to 8 months, or about 6 months to 9 months, or about 6 months to 10 months, or about 6 months to 11 months, or about 6 months to 12 months, or about 6 months to 13 months, or about 6 months to 14 months, or about 6 months to 15 months, or about 7 months to 8 months, or about 7 months to 9 months, or about 7 months to 10 months, or about 7 months to 11 months, or about 7 months to 12 months, or about 7 months to 13 months, or about 7 months to 14 months, or about 7 months to 15 months, or about 8 months to 9 months, or about 8 months to 10 months, or about 8 months to 11 months, or about 8 months to 12 months, or about 8 months to 13 months, or about 8 months to 14 months, or about 8 months to 15 months, or about 9 months to 10 months, or about 9 months to 11 months, or about 9 months to 12 months, or about 9 months to 13 months, or about 9 months to 14 months, or about 9 months to 15 months, or about 10 months to 11 months, or about 10 months to 12 months, or about 10 months to 13 months, or about 10 months to 14 months, or about 10 months to 15 months, or about 11 months to 12 months, or about 11 to 13 months, or about 11 to 14 months, or about 11 to 15 months, or about 12 to 13 months, or about 12 to 14 months, or about 12 to 15 months, or about 13 to 14 months, or about 13 to 15 months, or about 14 to 15 months. In some examples, long-term post-surgery is about 3 months to 12 months.

In some examples, the method comprises the design of multiplex primers for pancreatic-specific nucleic acids selected from tissue-enriched gene lists (such as pancreatic enriched gene list) according to databases known in the art (such as Human Protein Atlas). The inventors of the present disclosure subsequently amplified the pancreas-specific nucleic acid with the designed multiplex primers for quantification with real-time qPCR.

In some examples, the method comprises the design of multiplex primers for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more pancreatic-specific nucleic acid.

In some examples, the method comprises the design of multiplex primers for 14 pancreatic-specific nucleic acid. For example, the pancreatic-specific nucleic acid include genes such as, but is not limited to, Kallikrein 1 (KLK1), Chymotrypsinogen B1 (CTRB1), Endoplasmic Reticulum Protein 27 (ERP27), Islet Amyloid Polypeptide (IAPP), Serine Protease 1 (PRSS1), Chymotrypsin like Elastase 3A (CELA3A), Phospholipase A2 Group IB (PLA2G1B), Pancreatic triacylglycerol lipase (PNLIP), CUB and Zona Pellucida Like Domains 1 (CUZD1), Carboxypeptidase B1 (CPB1), Carboxypeptidase A1 (CPA1), Colipase (CLPS), insulin (INS), or Glucagon (GCG), and the like.

In some examples, the one or more pancreas-specific nucleic acid is detected using one or more of the primers provided in the following Table 1:

TABLE 1

| | | Reverse | | |
|---|---|---|---|---|
| Category | Targets | Primers | rh Primers | qPCR Primers |
| Pancreas | CTRB1 | | CAAGTACAACGCCAA | CAAGTACAACG |
| | Forward | | CAAGACrCCCTGG/3SpC3/ | CCAACAAGAC |
| | | | (SEQ ID NO: 55) | (SEQ ID NO: 1) |
| | CTRB1 | GTCCAGG | GTCCAGGCTCCATCC | GTCCAGGCTC |
| | Reverse | CTCCATC | TTTTrGGCAGT/3SpC3/ | CATCCTTTT |
| | | CTTTT | (SEQ ID NO: 56) | (SEQ ID NO: 2) |
| | | (SEQ ID | | |
| | | NO: 37) | | |
| | IAPP | | AATGGGCATCCTGAA | AATGGGCATC |
| | Forward | | GCTGrCAAGTT/3SpC3/ | CTGAAGCTG |
| | | | (SEQ ID NO: 57) | (SEQ ID NO: 3) |
| | IAPP | CATGTGG | CATGTGGCAGTGTTG | CATGTGGCAG |
| | Reverse | CAGTGTT | CATTTrCCGCTA/3SpC3/ | TGTTGCA (SEQ |
| | | GCA (SEQ | (SEQ ID NO: 58) | ID NO: 4) |
| | | ID NO: 38) | | |
| | CELA3A | | ACCATGGTGTGTGCT | ACCATGGTGT |
| | Forward | | GGAGrGGTACC/3SpC3/ | GTGCTGGAG |
| | | | (SEQ ID NO: 59) | (SEQ ID NO: 5) |
| | CELA3A | AACAAAG | AACAAAGCTGGTCAC | AACAAAGCTG |
| | Reverse | CTGGTCA | ACCGTrGGACCA/3SpC3/ | GTCACACC |
| | | CACC | (SEQ ID NO: 60) | (SEQ ID NO: 6) |
| | | (SEQ ID | | |
| | | NO: 39) | | |
| | PNLIP | | GGGAAAACAAATGAT | GGGAAAACAA |
| | Forward | | GTGGGrCCAGAG/3SpC3/ | ATGATGTGGG |
| | | | (SEQ ID NO: 61) | (SEQ ID NO: 7) |
| | PNLIP | TTTCCAG | TTTCCAGACAGTGTGA | TTTCCAGACAG |
| | Reverse | ACAGTGT | CAGATACCETTATAG/ | TGTGACAGATA |
| | | GACAGAT | 3SpC3/ | CC (SEQ ID NO: |
| | | ACC (SEQ | (SEQ ID NO: 62) | 8) |
| | | ID NO: 40) | | |
| | CPB1 | | TCAAGGCATATCTGAC | TCAAGGCATAT |
| | Forward | | AATCCArCTCGTT/3SpC3/ | CTGACAATCC |
| | | | (SEQ ID NO: 63) | (SEQ ID NO: 9) |
| | CPB1 | CAGTAGC | CAGTAGCTTTAGCCA | CAGTAGCTTTA |
| | Reverse | TTTAGCC | GGGCArTTCAAG/3SpC3/ | GCCAGGGCA |
| | | AGGGCA | (SEQ ID NO: 64) | (SEQ ID NO: 10) |
| | | (SEQ ID | | |
| | | NO: 41) | | |
| | CLPS | | TCCTGATCCTCCTGCT | TCCTGATCCTC |
| | Forward | | TGTCrGCCCTT/3SpC3/ | CTGCTTGTC |
| | | | (SEQ ID NO: 65) | (SEQ ID NO: 11) |
| | CLPS | TCATGCA | TCATGCAGAGCTCAC | TCATGCAGAG |
| | Reverse | GAGCTCA | CGTTrCTCCTC/3SpC3/ | CTCACCGTT |
| | | CCGTT | (SEQ ID NO: 66) | (SEQ ID NO: 12) |
| | | (SEQ ID | | |
| | | NO: 42) | | |
| | PRSS1 | | TGTGGTGGCTCCCTC | ATCAACGAACA |
| | Forward | | ATCAArCGAACT/3SpC3/ | GTGGGTGGT |
| | | | (SEQ ID NO: 67) | (SEQ ID NO: 13) |
| | PRSS1 | CACGCGG | CACGCGGGCGTTGAT | CTCATTCCCCT |
| | Reverse | GCGTTGA | TACTrGCACGA/3SpC3/ | CCAGGACTT |
| | | TTACT | (SEQ ID NO: 68) | (SEQ ID NO: 14) |
| | | (SEQ ID | | |
| | | NO: 43) | | |
| | PLA2G1B | | GAAACTCCTTGTGCTA | TTGTGCTAGCT |
| | Forward | | GCTGTGrCTGCTG/3SpC3/ | GTGCTGCTC |
| | | | (SEQ ID NO: 69) | (SEQ ID NO: 15) |
| | PLA2G1B | GTAGCAG | GTAGCAGCCGTAGTT | GCACTTGATCA |
| | Reverse | CCGTAGT | GTTGTrATTCCT/3SpC3/ | TTTTGCGG |
| | | TGTTGT | (SEQ ID NO: 70) | (SEQ ID NO: 16) |
| | | (SEQ ID | | |
| | | NO: 44) | | |
| | CUZD1 | | GGCTCATGCCATTGA | ATTCTCTCCTG |
| | Forward | | CCCTCrUTAATG/3SpC3/ | TTTGGCGG |
| | | | (SEQ ID NO: 71) | (SEQ ID NO: 17) |
| | CUZD1 | ACTGGGA | ACTGGGATTGAGTTG | TCATGGCTTTG |
| | Reverse | TTGAGTT | CAGGArUCATGC/3SpC3/ | TGGGTCTCT |
| | | GCAGGA | (SEQ ID NO: 72) | (SEQ ID NO: 18) |
| | | (SEQ ID | | |
| | | NO: 45) | | |
| | GCG | | CACAGGGCACATTCA | CACAGGGCAC |
| | Forward | | CCAGTrGACTAG/3SpC3/ | ATTCACCAGT |
| | | | (SEQ ID NO: 73) | (SEQ ID NO: 19) |
| | GCG | AATTCATC | AATTCATCGTGACGTT | AATTCATCGTG |
| | Reverse | GTGACGT | TGGCrAATGTA/3SpC3/ | ACGTTTGGC |
| | | TTGGC | (SEQ ID NO: 74) | (SEQ ID NO: 20) |
| | | (SEQ ID | | |
| | | NO: 46) | | |
| | CPA1 | | GCAAGATCCAGATTG | GCAAGATCCA |
| | Forward | | GCAACrACCTAG/3SpC3/ | GATTGGCAAC |
| | | | (SEQ ID NO: 75) | (SEQ ID NO: 21) |
| | CPA1 | CAGATGG | CAGATGGCTGGACGC | CAGATGGCTG |
| | Reverse | CTGGACG | TTACTrGCCCCG/3SpC3/ | GACGCTTACT |
| | | CTTACT | (SEQ ID NO: 76) | (SEQ ID NO: 22) |
| | | (SEQ ID | | |
| | | NO: 47) | | |
| | KLK1 | | TTTCCAGTGTGGGGG | CTCACAGCTG |
| | Forward | | CATCrCTGGTC/3SpC3/ | CTCATTGCAT |
| | | | (SEQ ID NO: 77) | (SEQ ID NO: 23) |
| | KLK1 | CATGTTG | CATGTTGAAGCCAGG | AACTGGGCTG |
| | Reverse | AAGCCAG | GTGTGrGGAAGG/3SpC3/ | TGTTTTCGTC |
| | | GGTGTG | (SEQ ID NO: 78) | (SEQ ID NO: 24) |
| | | (SEQ ID | | |
| | | NO: 48) | | |
| | ERP27 | | TGCCACCAAATTGAG | CCTCCACATG |
| | Forward | | CCGTrUTCATG/3SpC3/ | GTGACAGAGT |
| | | | (SEQ ID NO: 79) | ACA (SEQ ID |
| | | | | NO: 25) |
| | ERP27 | CTTGGCT | CTTGGCTGCCTTCTG | CTCTTCATACT |
| | Reverse | GCCTTCT | GTATCTrGTGCAA/3SpC3/ | CTGGGGAGGC |
| | | GGTATCT | (SEQ ID NO: 80) | (SEQ ID NO: 26) |
| | | (SEQ ID | | |
| | | NO: 49) | | |
| | INS | | CATCTGCTCCCTCTAC | CATCTGCTCCC |
| | Forward | | CArGCTGGG/3SpC3/ | TCTACCA (SEQ |
| | | | (SEQ ID NO: 81) | ID NO: 27) |
| | INS | CTTTATTC | CTTTATTCCATCTCTC | CTTTATTCCAT |
| | Reverse | CATCTCT | TCGGTrGCAGGT/3SpC3/ | CTCTCTCGGT |
| | | CTCGGT | (SEQ ID NO: 82) | (SEQ ID NO: 28) |
| | | (SEQ ID | | |
| | | NO: 50) | | |
| House | GAPDH | | ATGTTCGTCATGGGT | AGAAGTATGAC |
| keeping | Forward | | GTGrAACCAG/3SpC3/ | AACAGCCTCA |
| | | | (SEQ ID NO: 83) | (SEQ ID NO: 29) |
| | GAPDH | CATGAGT | CATGAGTCCTTCCAC | CCAAAGTTGTC |
| | Reverse | CCTTCCA | GATArCCAAAC/3SpC3/ | ATGGATGA |
| | | CGATA | (SEQ ID NO: 84) | (SEQ ID NO: 30) |
| | | (SEQ ID | | |
| | | NO: 51) | | |
| | ACTB | | AGAAGAGCTACGAGC | CATCACCATTG |
| | Forward | | TGCCTGArCGGCCC/3 | GCAATGA (SEQ |
| | | | SpC3/ (SEQ ID NO: 85) | ID NO: 31) |
| | ACTB | TGGAGTT | TGGAGTTGAAGGTAG | AAGGTAGTTTC |
| | Reverse | GAAGGTA | TTTCGrUGGATT/3SpC3/ | GTGGATGC |
| | | GTTTCG | (SEQ ID NO: 86) | (SEQ ID NO: 32) |
| | | (SEQ ID | | |
| | | NO: 52) | | |
| | RPS18 | | TATGCTCATGTGGTGT | AGACATTGACC |
| | Forward | | TGAGrGAAAGG/3SpC3/ | TCACCAAGA |
| | | | (SEQ ID NO: 87) | (SEQ ID NO: 33) |
| | RPS18 | AAGAACC | AAGAACCAGTCTGGG | TTGTACTGGCG |
| | Reverse | AGTCTGG | ATCTTrGTACTC/3SpC3/ | TGGATTC (SEQ |
| | | GATCTT | (SEQ ID NO: 88) | ID NO: 34) |
| | | (SEQ ID | | |
| | | NO: 53) | | |
| Control | LUC | | CTCTGATTAACGCCCA | CTCTGATTAAC |
| | Forward | | GCrGTTTTG/3SpC3/ | GCCCAGC |
| | | | (SEQ ID NO: 89) | (SEQ ID NO: 35) |
| | LUC | GCGGTCG | GCGGTCGGTAAAGTT | GCGGTCGGTA |
| | Reverse | GTAAAGT | GTTrCCATTA/3SpC3/ | AAGTTGTT |
| | | TGTT (SEQ | (SEQ ID NO: 90) | (SEQ ID NO: 36) |
| | | ID NO: 54) | | |

In another aspect, there is provided a primer or a primer set comprising one or more primers having a sequence listed in Table 1, Table 2, Table 3 and/or Table 4, or a part thereof, or a sequence sharing at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto.

In some examples, the method comprises the extraction of a pancreatic-specific nucleic acid (such as cfmRNA) from a sample (such as plasma). In some examples, residual nucleic acid (such as DNA in the cfmRNA) was digested using an enzyme (such as RNase-free DNase I). In some examples, the extracted nucleic acid (such as cfmRNA) was purified.

In some examples, the method comprises yielding of at least 0 to 50 μl of pancreatic-specific nucleic acid per sample. For example, the yield of pancreatic-specific nucleic acid per sample may include, but is not limited to, 1 μl, 2 μl, 3 μl, 4 μl, 5 μl, 6 μl, 7 μl, 8 μl, 9 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl, 43 μl, 44 μl, 45 μl, 46 μl, 47 μl, 48 μl, 49 μl, or 50 μl, and the like. In some examples, the method comprises yielding 24 μl of pancreatic-specific cfmRNA per sample.

In some examples, the annealing step precedes the reverse transcription and amplification cycles.

In some examples, the method further comprises adding a control nucleic acid to the sample. In some examples, the control nucleic acid is added to the sample at a constant amount to thereby normalizes of the amplification efficiency across a plurality of samples, optionally the control nucleic acid is added to the sample at about 10² to 10¹⁰ copies.

Advantageously, spiking of the sample with a control nucleic acid such as a luciferase RNA allows for the normalization of any technical variability arising from the amplification process. This leads to more accurate detection and/or quantification of the target nucleic acid.

A suitable housekeeping gene may be included during amplification for quality control. Examples of suitable housekeeping genes include, but are not limited to, ACTB, GAPDH, RPS18 and luciferase (LUC) genes.

In various embodiments therefore, the method comprises performing one of the steps of the amplification process/PCR (e.g., rhPCR) at approximately the melting temperature of the PCR products or the dsDNA amplicons. In various embodiments, the method comprises performing the denaturing step of the amplification process/PCR (e.g., rhPCR) at approximately the melting temperature of the PCR products or the dsDNA amplicons. The melting temperature of the PCR products or the dsDNA amplicons may be obtained by plotting a melting curve. In various embodiments, the denaturing step of the amplification process/PCR is carried out at a temperature of from about 80° C. to about 100° C., from about 90° C. to about 100° C., from about 92° C. to about 96° C., or from about 93° C. to about 95° C. In various embodiments, the denaturing step of the amplification process/PCR is carried out at a temperature of about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C. or about 100° C. In one embodiment, the denaturing step of the amplification process/PCR is carried out at a temperature of about 94° C.

In various embodiments, the melting temperature is held for at least about 5 seconds, at least about 6 seconds, at least about 7 seconds, at least about 8 seconds, at least about 9 seconds, at least about 10 seconds, at least about 11 seconds, at least about 12 seconds, at least about 13 seconds, at least about 14 seconds or at least about 15 seconds during the amplification process/PCR.

In various embodiments, denaturing is carried out from about 10 to about 30 cycles, or from about 15 to about 25 cycles. In various embodiments, denaturing is carried out for at least about 10 cycles, at least about 11 cycles, at least about 12 cycles, at least about 13 cycles, at least about 14 cycles, at least about 15 cycles, at least about 16 cycles, at least about 17 cycles, at least about 18 cycles, at least about 19 cycles, at least about 20 cycles, at least about 21 cycles, at least about 22 cycles, at least about 23 cycles, at least about 24 cycles, at least about 25 cycles, at least about 26 cycles, at least about 27 cycles, at least about 28 cycles, at least about 29 cycles or at least about 30 cycles.

In various embodiments therefore, the method comprises a multiplex method. In some examples, the method further comprises performing one or more of the following: RNaseH2-dependent PCR, emulsion PCR, and/or CoT PCR. In some examples, the method may comprise quantifying at least two pancreas-associated RNA (i.e., the method is a multiplex method).

The inventors of the present disclosure describe the use of a circulating cfmRNA based multiplex quantitative polymerase chain reaction (qPCR) assay to selectively amplify and quantify circulating pancreatic-specific transcripts levels within the plasma In this targeted multiplex screen of 14 selected pancreatic specific circulating transcripts, the inventors of the present disclosure identified 13 pancreatic specific transcripts that can be amplified from the plasma.

In some examples, the method further comprises a reverse transcription of the one or more pancreatic-specific nucleic acid after annealing step. In some examples, the method further comprises subjecting the pancreatic-specific nucleic acid to reverse transcription. In some examples, the method further comprises a reverse transcription of the pancreatic-specific nucleic acid after annealing step.

In some examples, the method comprises contacting the pancreatic-specific nucleic acid with a reverse transcription agent comprising a reverse transcriptase.

In some examples, the method further comprises reverse transcribing the pancreas-associated RNA.

In various embodiments, the primers used for reverse transcription comprises a sequence listed in Table 2 below or a part thereof, or a sequence sharing at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto.

TABLE 2

| Category | Targets | Reverse Primers |
|---|---|---|
| Pancreas | CTRB1 | GTCCAGGCTCCATCCTTTT |
| | Reverse | (SEQ ID NO: 37) |
| | IAPP Reverse | CATGTGGCAGTGTTGCA |
| | | (SEQ ID NO: 38) |
| | CELA3A | AACAAAGCTGGTCACACC |
| | Reverse | (SEQ ID NO: 39) |
| | PNLIP | TTTCCAGACAGTGTGACAGATACC |
| | Reverse | (SEQ ID NO: 40) |
| | CPB1 | CAGTAGCTTTAGCCAGGGCA |
| | Reverse | (SEQ ID NO: 41) |
| | CLPS | TCATGCAGAGCTCACCGTT |
| | Reverse | (SEQ ID NO: 42) |
| | PRSS1 | CACGCGGGCGTTGATTACT |
| | Reverse | (SEQ ID NO: 43) |
| | PLA2G1B | GTAGCAGCCGTAGTTGTTGT |
| | Reverse | (SEQ ID NO: 44) |
| | CUZD1 | ACTGGGATTGAGTTGCAGGA |
| | Reverse | (SEQ ID NO: 45) |
| | GCG Reverse | AATTCATCGTGACGTTTGGC |
| | | (SEQ ID NO: 46) |
| | CPA1 | CAGATGGCTGGACGCTTACT |
| | Reverse | (SEQ ID NO: 47) |
| | KLK1 Reverse | CATGTTGAAGCCAGGGTGTG |
| | | (SEQ ID NO: 48) |
| | ERP27 | CTTGGCTGCCTTCTGGTATCT |
| | Reverse | (SEQ ID NO: 49) |
| | INS Reverse | CTTTATTCCATCTCTCTCGGT |
| | | (SEQ ID NO: 50) |
| House- | GAPDH | CATGAGTCCTTCCACGATA |
| keeping | Reverse | (SEQ ID NO: 51) |
| | ACTB | TGGAGTTGAAGGTAGTTTCG |
| | Reverse | (SEQ ID NO: 52) |
| | RPS18 | AAGAACCAGTCTGGGATCTT |
| | Reverse | (SEQ ID NO: 53) |
| Control | LUC Reverse | GCGGTCGGTAAAGTTGTT |
| | | (SEQ ID NO: 54) |

In some examples, the method further comprises inactivation of the reverse transcriptase.

In some examples, the method further comprises subjecting the products obtained from the preceding step (e.g., the cDNA obtained after reverse transcribing the pancreas-associated RNA) to RNaseH2-dependent polymerase chain reaction (rhPCR).

In one example, the primers used for the rhPCR comprises exon-spanning rhPCR primers (rhPrimers). The rhPrimer may comprise an RNA base. In one example, a RNase H2 enzyme such as a thermostable RNase H2 enzyme is used in the rhPCR. Advantageously, in rhPCR, DNA synthesis may only occur when there is prefect complementarity between a primer and the target polynucleotide (e.g., the cDNA obtained after reverse transcribing the pancreas-associated RNA) intended to be amplified. Thus, improved specificity of the priming event allows for accurate and targeted amplification.

In some examples, the primers used for the rhPCR comprises one or more of the following: a first matching site comprising bases that are complementary to a target polynucleotide/template or portions thereof, a cleavage site, a second or further matching site comprising bases that are complementary to a target polynucleotide/template or portions thereof, a mismatch site and a blocking group.

In various embodiments, the first matching site comprises at least about 6, at least about 8, at least about 10 at least about 12 DNA bases that are complementary to the target polynucleotide/template or portions thereof. The first matching site may be located at a terminal end such as the 5′ end of the primer. The first matching site may be located upstream of the cleavage site. In various embodiments, the cleavage site comprises a single RNA base. In various embodiments, the second or further matching site comprises about 1, about 2, about 3, about 4, about 5, about 6, about 7, about 8, about 9 or about 10 DNA bases that are complementary to the target polynucleotide/template or portions thereof. In one embodiment, the second matching site comprises about 4 DNA bases that are complementary to the target polynucleotide/template or portions thereof. In various embodiments, the mismatch site comprises a mismatch DNA base. The blocking group may comprise a C3 spacer, although not limited as such. The blocking group may be located at a terminal end such as the 3′ end or a proximal terminal end of the primer. In some embodiments, the blocking group is located at the 3′ end of the primer.

In various embodiments, the primer may have the following structure:
5′-D₁-R-D₂-M-x-3′; or
5′-D₁-R-D₂-X-D₃-M-3′
   wherein each of D₁, D₂ and D₃ represents a matching site comprising DNA bases that are complementary to the target polynucleotide/template or portions thereof, R represents a cleavage site, M represents a mismatch site and x represents a blocking group.

In some embodiments, at least one of the forward and reverse primers comprises the blocking group. In some embodiments, both the forward and reverse primers comprise the blocking group.

In various embodiments, the primers comprises a sequence listed in Table 3 below or a part thereof, or a sequence sharing at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto.

TABLE 3

| Category | Targets | rh Primers |
|---|---|---|
| Pancreas | CTRB1 | CAAGTACAACGCCAACAAGACrCCCTGG/3SpC3/ |
| | Forward | (SEQ ID NO: 55) |
| | CTRB1 | GTCCAGGCTCCATCCTTTTrGGCAGT/3SpC3/ (SEQ |
| | Reverse | ID NO: 56) |
| | IAPP Forward | AATGGGCATCCTGAAGCTGrCAAGTT/3SpC3/ (SEQ |
| | | ID NO: 57) |
| | IAPP Reverse | CATGTGGCAGTGTTGCATTTrCCGCTA/3SpC3/ |
| | | (SEQ ID NO: 58) |
| | CELA3A | ACCATGGTGTGTGCTGGAGrGGTACC/3SpC3/ |
| | Forward | (SEQ ID NO: 59) |
| | CELA3A | AACAAAGCTGGTCACACCGTrGGACCA/3SpC3/ |
| | Reverse | (SEQ ID NO: 60) |
| | PNLIP | GGGAAAACAAATGATGTGGGrCCAGAG/3SpC3/ |
| | Forward | (SEQ ID NO: 61) |
| | PNLIP | TTTCCAGACAGTGTGACAGATACCETTATAG/3SpC3/ |
| | Reverse | (SEQ ID NO: 62) |
| | CPB1 | TCAAGGCATATCTGACAATCCArCTCGTT/3SpC3/ |
| | Forward | (SEQ ID NO: 63) |
| | CPB1 | CAGTAGCTTTAGCCAGGGCArTTCAAG/3SpC3/ |
| | Reverse | (SEQ ID NO: 64) |
| | CLPS | TCCTGATCCTCCTGCTTGTCrGCCCTT/3SpC3/ |
| | Forward | (SEQ ID NO: 65) |
| | CLPS | TCATGCAGAGCTCACCGTTrCTCCTC/3SpC3/ (SEQ |
| | Reverse | ID NO: 66) |
| | PRSS1 | TGTGGTGGCTCCCTCATCAArCGAACT/3SpC3/ |
| | Forward | (SEQ ID NO: 67) |
| | PRSS1 | CACGCGGGCGTTGATTACTrGCACGA/3SpC3/ (SEQ |
| | Reverse | ID NO: 68) |
| | PLA2G1B | GAAACTCCTTGTGCTAGCTGTGrCTGCTG/3SpC3/ |
| | Forward | (SEQ ID NO: 69) |
| | PLA2G1B | GTAGCAGCCGTAGTTGTTGTrATTCCT/3SpC3/ |
| | Reverse | (SEQ ID NO: 70) |
| | CUZD1 | GGCTCATGCCATTGACCCTCrUTAATG/3SpC3/ |
| | Forward | (SEQ ID NO: 71) |
| | CUZD1 | ACTGGGATTGAGTTGCAGGArUCATGC/3SpC3/ |
| | Reverse | (SEQ ID NO: 72) |
| | GCG Forward | CACAGGGCACATTCACCAGTrGACTAG/3SpC3/ |
| | | (SEQ ID NO: 73) |
| | GCG Reverse | AATTCATCGTGACGTTTGGCrAATGTA/3SpC3/ |
| | | (SEQ ID NO: 74) |
| | CPA1 | GCAAGATCCAGATTGGCAACrACCTAG/3SpC3/ |
| | Forward | (SEQ ID NO: 75) |
| | CPA1 | CAGATGGCTGGACGCTTACTrGCCCCG/3SpC3/ |
| | Reverse | (SEQ ID NO: 76) |
| | KLK1 Forward | TTTCCAGTGTGGGGGCATCrCTGGTC/3SpC3/ (SEQ |
| | | ID NO: 77) |
| | KLK1 Reverse | CATGTTGAAGCCAGGGTGTGrGGAAGG/3SpC3/ |
| | | (SEQ ID NO: 78) |
| | ERP27 | TGCCACCAAATTGAGCCGTrUTCATG/3SpC3/ (SEQ |
| | Forward | ID NO: 79) |
| | ERP27 | CTTGGCTGCCTTCTGGTATCTrGTGCAA/3SpC3/ |
| | Reverse | (SEQ ID NO: 80) |
| | INS Forward | CATCTGCTCCCTCTACCArGCTGGG/3SpC3/ (SEQ |
| | | ID NO: 81) |
| | INS Reverse | CTTTATTCCATCTCTCTCGGTrGCAGGT/3SpC3/ |
| | | (SEQ ID NO: 82) |
| House- | GAPDH | ATGTTCGTCATGGGTGTGrAACCAG/3SpC3/ (SEQ |
| keeping | Forward | ID NO: 83) |
| | GAPDH | CATGAGTCCTTCCACGATArCCAAAC/3SpC3/ (SEQ |
| | Reverse | ID NO: 84) |
| | ACTB | AGAAGAGCTACGAGCTGCCTGArCGGCCC/3SpC3/ |
| | Forward | (SEQ ID NO: 85) |
| | ACTB | TGGAGTTGAAGGTAGTTTCGrUGGATT/3SpC3/ |
| | Reverse | (SEQ ID NO: 86) |
| | RPS18 | TATGCTCATGTGGTGTTGAGrGAAAGG/3SpC3/ |
| | Forward | (SEQ ID NO: 87) |
| | RPS18 | AAGAACCAGTCTGGGATCTTrGTACTC/3SpC3/ |
| | Reverse | (SEQ ID NO: 88) |
| Control | LUC Forward | CTCTGATTAACGCCCAGCrGTTTTG/3SpC3/ (SEQ |
| | | ID NO: 89) |
| | LUC Reverse | GCGGTCGGTAAAGTTGTTrCCATTA/3SpC3/ (SEQ |
| | | ID NO: 90) |

In some examples, the rhPCR is carried out in an oil-water emulsion.

Surfactants may be added to a PCR reaction mixture to create an oil-water emulsion. In one example, a fluorosurfactant is added to a PCR reaction mixture to create an oil-water emulsion. The resulting amplicons may be subsequently recovered by freezing the oil-water emulsion.

Advantageously, the PCR reaction mixture is compartmentalized when carrying out the rhPCR in an oil-water emulsion and hence this reduces the formation of any unwanted chimeric products.

In some examples, the amplification step of the nucleic acid is performed in the presence of three parts surfactant to one part amplification mixture.

In some examples, method comprises two amplification steps.

In some examples, the amplification step of the nucleic acid is performed in the presence of three parts surfactant to one part amplification mixture. In some examples, the amplification step is performed in an emulsion mixture. The emulsion mixture is made up of 1 to 10 parts of surfactant with 1 to 5 parts of amplification mixture, or 1 part of surfactant with 1 part of amplification mixture, or 2 parts of surfactant with 1 part of amplification mixture, or 3 parts of surfactant with 1 part of amplification mixture, or 4 parts of surfactant with 1 part of amplification mixture, or 5 parts of surfactant with 1 part of amplification mixture, or 6 parts of surfactant with 1 part of amplification mixture, or 7 parts of surfactant with 1 part of amplification mixture, or 8 parts of surfactant with 1 part of amplification mixture, or 9 parts of surfactant with 1 part of amplification mixture, or 10 parts of surfactant with 1 part of amplification mixture, or 3 parts of surfactant with 2 parts of amplification mixture, or 4 parts of surfactant with 2 parts of amplification mixture, or 5 parts of surfactant with 2 parts of amplification mixture, or 6 parts of surfactant with 2 parts of amplification mixture, or 7 parts of surfactant with 2 parts of amplification mixture, or 8 parts of surfactant with 2 parts of amplification mixture, or 9 parts of surfactant with 2 parts of amplification mixture, or 10 parts of surfactant with 2 parts of amplification mixture. In some examples, the emulsion is made up of 3 parts of surfactant with 1 part of amplification mixture. That is, the emulsion is made up of 3 parts of surfactant with 1 part of amplification mixture, when the amplification mixture is 10 μL, the surfactant is 30 μL, to thereby provide a total of 40 μL of emulsion mixture.

In some examples, the surfactant may be used at about 1% (w/w) to about 15% (w/w), or about 2, 3, 4, 5, 6, 7, 8, 9, 10, 11, 12, 13, 14, or 15% (w/w). In some examples, the surfactant may be 10% (w/w) of fluorosurfactant.

In some examples, method comprises two amplification steps.

In some examples, the method comprises 2, or 3, or 4, or 5 amplification steps. In some examples, the amplification cycle or step is repeated two times to five times. In some examples, the amplification step is repeated two times, or three times, or four times, or five times, or more. In some examples, the amplification step is repeated two times (i.e., two amplification cycles).

In some examples, the method may comprise adding an amplification (e.g., PCR) mixture to the pancreatic-specific nucleic acid. In some examples, the mixture may comprise a DNA polymerase, an rhPCR mixture of the pancreatic-specific nucleic acid, and an RNase (such as an RNase H2 enzyme).

In some examples, the method comprises generating an emulsion by adding 3 parts of surfactant to 1 part of PCR reaction mixture. In some examples, the method comprises mixing (such as vortexing) the emulsion generated until cloudy and uniform. In some examples, the amplification step may be a thermocycling reaction with enzyme activation, denaturation, annealing, and extension.

In some examples, the method comprises transferring the top fraction of the reaction mixture to a fresh tube. In some examples, the method further comprises topping up the fraction recovered with the same amount of polymerase (such as Taq polymerase) and RNase enzyme (such as RNase H2 enzyme) as used in the preceding PCR reaction.

In some examples, the method further comprises a step of freeze and thawing the amplified mixture.

In some examples, the method further comprises a step of freeze and thawing the amplified mixture between the one or more amplification steps

In some examples, the method further comprises removing residual primers with an enzyme, followed by enzyme inactivation.

In some examples, the method further comprises subjecting the products obtained from the preceding step (e.g., the amplicons recovered from the emulsion) to CoT PCR.

In some examples, the method comprises a subsequent amplification step (e.g., a second or third or more thermocycling reactions) with enzyme activation, denaturation, hybridization, annealing and extension.

Advantageously, CoT PCR enrichment may preferentially amplify rare amplicons over abundant ones by taking advantage of the CoT effect.

In a comparative example, a combination of emulsion rhPCR and CoT is shown to decrease the number of PCR cycles required for detection of a target polynucleotide as compared to emulsion rhPCR without CoT. Thus, a combination of emulsion rhPCR and CoT may advantageously increase a sensitivity of embodiments of the method in determining, detecting or quantifying a pancreas-associated polynucleotide. This allows embodiments of the method to detect or quantify low levels of pancreas-associated polynucleotide in a subject, which may not be possible otherwise.

Without wishing to be bound by theory, it is believed that the Cot effect is described by a faster rate of re-association of higher abundance single stranded DNA (ssDNA) to double-stranded DNA (dsDNA). Using this phenomenon, one way to enrich for rare target is to allow the higher abundant ssDNA to reassociate to dsDNA and then follow up with a method to remove the dsDNA to achieve removal of abundant dsDNA. In the present disclosure however, embodiments of the method do not involve removing the abundant dsDNA that was amplified (e.g., by rhPCR). Instead, the less abundant DNA in the reaction is allowed to have a higher probability of amplified. To achieve this, the CoT phenomenon may be implemented during amplification (e.g., during rhPCR) by adjusting/controlling the thermal cycling profile of the amplification process (e.g., rhPCR). The PCR reaction mix (e.g., the rhPCR reaction mix) may be held at the melting temperature of the dsDNA amplicons during the denaturing step of the PCR (e.g., rhPCR). Without wishing to be bound by theory, it is believed that, when the PCR reaction is being held at the melting temperature of the dsDNA amplicon, the CoT effect kicks in: the abundant dsDNA preferentially remains double stranded, and only dsDNA amplicons at low concentration will dissociate. These dissociated ssDNA are the only ones accessible to primers in the subsequent annealing and extension step which completes the PCR. By repeating this every cycle (i.e., holding the PCR reaction at the melting temperature of the dsDNA amplicon at the denaturing step), the initial low abundance amplicons will amplify up to a point where it becomes suitably abundant and joins other high abundance amplicons and be inhibited from disassociation, which allows for other remaining low abundance amplicons to be amplified.

In some examples, the method further comprises a step of quantifying the amount of one or more pancreatic-specific nucleic acid present in the sample and/or sequencing the target nucleic acid in the sample.

In some examples, the method of the present disclosure may be adaptable to include processing where amplified cDNA exhibits compatibility for downstream further processing. This is because the method of the present disclosure advantageously provides an adaptable end point where amplified cDNA exhibit compatibility for downstream quantification using methods known in the art. For example, the cDNA as amplified by the method as disclosed herein may be used in further steps of quantifying the amount of target nucleic acid by performing quantitative real-time PCR, next generation sequencing, UV absorbance with spectrophotometer, fluorescence dyes, agarose gel electrophoresis, microfluidic capillary electrophoresis, diphenylamine method, droplet digital PCR, and the like.

In some examples, the pancreas-associated nucleic acid may be determined, detected or quantified by use of sequencing techniques. Examples of sequencing techniques include next-generation sequencing, nanopore sequencing, amplicon-based sequencing, paired-end sequencing, Sanger sequencing etc. In various embodiments, deep sequencing is carried out. In various embodiments, next-generation sequencing is carried out. In some examples, the determining, detecting or quantifying the pancreas-associated nucleic acid comprises performing quantitative polymerase chain reaction (qPCR). In some examples, the expression of the targeted nucleic acid may be monitored at different time points using qPCR.

In various embodiments, the primers used for qPCR comprises a sequence listed in Table 4 below or a part thereof, or a sequence sharing at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto.

TABLE 4

| Category | Targets | qPCR Primers |
|---|---|---|
| Pancreas | CTRB1 Forward | CAAGTACAACGCCAACAAGAC (SEQ ID |
| | | NO: 1) |
| | CTRB1 Reverse | GTCCAGGCTCCATCCTTTT (SEQ ID NO: |
| | | 2) |
| | IAPP Forward | AATGGGCATCCTGAAGCTG (SEQ ID NO: |
| | | 3) |
| | IAPP Reverse | CATGTGGCAGTGTTGCA (SEQ ID NO: 4) |
| | CELA3A | ACCATGGTGTGTGCTGGAG (SEQ ID NO: |
| | Forward | 5) |
| | CELA3A | AACAAAGCTGGTCACACC (SEQ ID NO: 6) |
| | Reverse | |
| | PNLIP Forward | GGGAAAACAAATGATGTGGG (SEQ ID |
| | | NO: 7) |
| | PNLIP Reverse | TTTCCAGACAGTGTGACAGATACC (SEQ |
| | | ID NO: 8) |
| | CPB1 Forward | TCAAGGCATATCTGACAATCC (SEQ ID |
| | | NO: 9) |
| | CPB1 Reverse | CAGTAGCTTTAGCCAGGGCA (SEQ ID NO: |
| | | 10) |
| | CLPS Forward | TCCTGATCCTCCTGCTTGTC (SEQ ID NO: |
| | | 11) |
| | CLPS Reverse | TCATGCAGAGCTCACCGTT (SEQ ID NO: |
| | | 12) |
| | PRSS1 Forward | ATCAACGAACAGTGGGTGGT (SEQ ID NO: |
| | | 13) |
| | PRSS1 Reverse | CTCATTCCCCTCCAGGACTT (SEQ ID NO: |
| | | 14) |
| | PLA2G1B | TTGTGCTAGCTGTGCTGCTC (SEQ ID NO: |
| | Forward | 15) |
| | PLA2G1B | GCACTTGATCATTTTGCGG (SEQ ID NO: |
| | Reverse | 16) |
| | CUZD1 Forward | ATTCTCTCCTGTTTGGCGG (SEQ ID NO: |
| | | 17) |
| | CUZD1 Reverse | TCATGGCTTTGTGGGTCTCT (SEQ ID NO: |
| | | 18) |
| | GCG Forward | CACAGGGCACATTCACCAGT (SEQ ID NO: |
| | | 19) |
| | GCG Reverse | AATTCATCGTGACGTTTGGC (SEQ ID NO: |
| | | 20) |
| | CPA1 Forward | GCAAGATCCAGATTGGCAAC (SEQ ID NO: |
| | | 21) |
| | CPA1 Reverse | CAGATGGCTGGACGCTTACT (SEQ ID NO: |
| | | 22 |
| | KLK1 Forward | CTCACAGCTGCTCATTGCAT (SEQ ID NO: |
| | | 23) |
| | KLK1 Reverse | AACTGGGCTGTGTTTTCGTC (SEQ ID NO: |
| | | 24) |
| | ERP27 Forward | CCTCCACATGGTGACAGAGTACA (SEQ |
| | | ID NO: 25) |
| | ERP27 Reverse | CTCTTCATACTCTGGGGAGGC (SEQ ID |
| | | NO: 26) |
| | INS Forward | CATCTGCTCCCTCTACCA (SEQ ID NO: 27) |
| | INS Reverse | CTTTATTCCATCTCTCTCGGT (SEQ ID NO: |
| | | 28) |
| House- | GAPDH Forward | AGAAGTATGACAACAGCCTCA (SEQ ID |
| keeping | | NO: 29) |
| | GAPDH Reverse | CCAAAGTTGTCATGGATGA (SEQ ID NO: |
| | | 30) |
| | ACTB Forward | CATCACCATTGGCAATGA (SEQ ID NO: 31) |
| | ACTB Reverse | AAGGTAGTTTCGTGGATGC (SEQ ID NO: |
| | | 32) |
| | RPS18 Forward | AGACATTGACCTCACCAAGA (SEQ ID NO: |
| | | 33) |
| | RPS18 Reverse | TTGTACTGGCGTGGATTC (SEQ ID NO: |
| | | 34) |
| Control | LUC Forward | CTCTGATTAACGCCCAGC (SEQ ID NO: |
| | | 35) |
| | LUC Reverse | GCGGTCGGTAAAGTTGTT (SEQ ID NO: |
| | | 36) |

In various embodiments, the method further comprises generating and/or analysing melting curves and/or amplification plots of the PCR.

As shown in the experimental data, when quantifying the amplicons obtained in the short-term post-surgery (2 weeks to 1 month) and long-term (3 to 12 months), the inventors of the present disclosure observed a consistent reduction of circulating GCG transcripts during short term post-surgery. Across the cohort, GCG cfmRNA levels correlated significantly with common metrics of improvement following bariatric surgery such as: haemoglobin A1c levels (R: −0.41, P-value: 0.0039) and percentage of excess weight loss (R: 0.29, P-value: 0.046).

In yet another aspect, there is provided a kit for assessing/monitoring the health (e.g., a pancreatic health or a metabolic health) of a subject, the kit comprising one or more of the following: one or more primers/primer set as described in the preceding AS, a control polynucleotide as described herein (e.g., a luciferase RNA); medium/buffer/solution; water (e.g., distilled water, RNase-free water and/or nuclease-free water); deoxynucleotide triphosphates (dNTPs) or modified nucleotides or the like, RNase inhibitor; one or more polymerases (e.g., a DNA polymerase and/or a reverse transcriptase); an RNase H2 enzyme; a surfactant (e.g., a fluorosurfactant) and a control dye (e.g., SYBR Green I and/or ROX).

In yet another aspect, there is provided a method or a product as described herein.

In some examples, there is provided a method of amplification of a target nucleic acid, the method comprising annealing the target nucleic acid in the presence of a control nucleic acid, and subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

In some examples, the target nucleic acid may be a pancreatic-specific nucleic acid.

In some examples, the amplification step of the nucleic acid is performed in the presence of three parts surfactant to one part amplification mixture. In some examples, the amplification step is performed in an emulsion mixture. The emulsion mixture is made up of 1 to 10 parts of surfactant with 1 to 5 parts of amplification mixture, or 1 part of surfactant with 1 part of amplification mixture, or 2 parts of surfactant with 1 part of amplification mixture, or 3 parts of surfactant with 1 part of amplification mixture, or 4 parts of surfactant with 1 part of amplification mixture, or 5 parts of surfactant with 1 part of amplification mixture, or 6 parts of surfactant with 1 part of amplification mixture, or 7 parts of surfactant with 1 part of amplification mixture, or 8 parts of surfactant with 1 part of amplification mixture, or 9 parts of surfactant with 1 part of amplification mixture, or 10 parts of surfactant with 1 part of amplification mixture, or 3 parts of surfactant with 2 parts of amplification mixture, or 4 parts of surfactant with 2 parts of amplification mixture, or 5 parts of surfactant with 2 parts of amplification mixture, or 6 parts of surfactant with 2 parts of amplification mixture, or 7 parts of surfactant with 2 parts of amplification mixture, or 8 parts of surfactant with 2 parts of amplification mixture, or 9 parts of surfactant with 2 parts of amplification mixture, or 10 parts of surfactant with 2 parts of amplification mixture. In some examples, the emulsion is made up of 3 parts of surfactant with 1 part of amplification mixture. That is, the emulsion is made up of 3 parts of surfactant with 1 part of amplification mixture, when the amplification mixture is 10 μL, the surfactant is 30 μL, to thereby provide a total of 40 μL of emulsion mixture.

In some examples, the surfactant may be used at about 1% (w/w) to about 15% (w/w), or about 2, 3, 4, 5, 6, 7, 8, 9, 10, 11, 12, 13, 14, or 15% (w/w). In some examples, the surfactant may be 10% (w/w) of fluorosurfactant.

In some examples, method comprises two amplification steps.

In some examples, the method comprises 2, or 3, or 4, or 5 amplification steps. In some examples, the amplification cycle or step is repeated two times to five times. In some examples, the amplification step is repeated two times, or three times, or four times, or five times, or more. In some examples, the amplification step is repeated two times (i.e. two amplification cycles).

In some examples, the method further comprises a step of freeze and thawing the amplified mixture.

In some examples, wherein the method further comprises a step of freeze and thawing the amplified mixture between the one or more amplification steps.

In some examples, the freeze and thawing step may be referred to as the emulsion breaking step. The inventors of the present disclosure found freeze thawing the emulsion PCR product advantageously provides for a robust, non-chemical based method of recovering the emulsion PCR product.

In some examples, the step of freezing comprises subjecting the mixture to a condition that freezes the mixture to a solid state. For example, the step of freezing subjects the mixture to a below freezing conditions. In some examples, the method comprises the step of freezing the mixture to 0° C. to −100° C., or to −50° C., or to −60° C., or to −70° C., or to −80° C. or to −90° C., or to −100° C. In some examples, the method comprises the step of freezing the mixture to −80° C.

In some examples, the method comprises freezing the reaction mixture for 0.5 hour to overnight. In some examples, the method comprises freezing the reaction mixture for 0.5 hour, or 1 hour, or 1.5 hour, or 2 hours, or 2.5 hours, or 3 hours, or 3.5 hours, o 4 hours, or 4.5 hours, or 5 hours, or 6 hours, or 7 hours, or 8 hours, or 9 hours, or overnight.

In some examples, the step of thawing comprises subjecting the mixture to a condition that allows the mixture to gain heat and change to a liquid state. For example, the step of thawing subjects the mixture to an above 0° C. conditions. In some examples, the step of thawing subjects the mixture to room temperature condition, for example from about 0° C. to about 40° C.

In some examples, the method may comprise adding an amplification (e.g. PCR) mixture to the target nucleic acid. In some examples, the mixture may comprise a DNA polymerase, a dNTP mixture, a cofactor (such as Magnesium Chloride), an rhPCR mixture of the target nucleic acid, and an RNase (such as an RNase H2 enzyme).

In some examples, the method comprises generating an emulsion by adding 3 parts of surfactant to 1 part of PCR reaction mixture. In some examples, the method comprises mixing (such as vortexing) the emulsion generated until uniform turbidity. In some examples, the amplification step may be a thermocycling reaction with enzyme activation, denaturation, annealing, and extension.

In some examples, the method comprises freezing the reaction mixture for 1 hour before thawing at room temperature. In some examples, the freeze thaw is performed between each amplification (thermocycling) step. In some examples, the method comprises transferring the top fraction of the reaction mixture to a fresh tube. In some examples, the method further comprises topping up the fraction recovered with the same amount of polymerase (such as Taq polymerase) and RNase enzyme (such as RNase H2 enzyme) as used in the preceding PCR reaction.

In some examples, the method comprises a subsequent amplification step (e.g. a second or third or more thermocycling reactions) with enzyme activation, denaturation, hybridization, annealing and extension. In some examples, the method further comprises removing residual primers with an enzyme, followed by enzyme inactivation.

In some examples, the oligonucleotide primer and/or probe capable of hybridizing with the target nucleic acid comprises a 5′ end of a functional primer, a cleavage site, one or more matching DNA bases, and one or more mismatch DNA base with one or more blocking group at the 3′ end.

In some examples, the primer may comprise about 10 to 40 bases, or 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 35 bases, or 40 bases.

In some examples, the functional primer may be about 16 to 24 bases, or about 16, 17, 18, 19, 20, 21, 22, 23, or 24 bases. In some examples, the reverse primers may comprise about 15 to 18 bases, or about 15, 16, 17, 18, 19, 20, 21, 22, 23, or 24 bases. In some examples, the qPCR primers may comprise about 15 to 20 bases, or 15, 17, 18, 19, 20, 21, 22, 23, or 24 bases

In some examples, the cleavage site is one or more RNA residue. In some examples, the cleavage site may comprise 2, 3, or 4 RNA residue. In some examples, the cleavage site is a single RNA residue or one RNA residue. In some examples, the cleavage site may be one or more of rU, rC, rG, or rA. In some examples, the cleavage site may be one of rC, rG, or rA.

In some examples, the cleavage site is one or more RNA residues.

In some examples, oligonucleotide primer and/or probe capable of hybridizing with the target nucleic acid comprises a 5′ end of a functional primer, a cleavage site consisting of one or more RNA residue, one or more matching DNA bases, and one or more mismatch DNA base with one or more blocking group at the 3′ end.

In some examples, the method comprises the step of cleaving the oligonucleotide primer and/or probe with an RNase enzyme.

In some examples, the cleavage site is cleaved by RNase H2 enzyme.

In some examples, the cleavage of the RNA residue releases the blocking group.

In some examples, the one or more matching DNA bases may comprise 1 DNA base, 2 DNA bases, 3 DNA bases, 4 DNA bases, 5 DNA bases, 6 DNA bases, 7 DNA bases, 8 DNA bases, 9 DNA bases, or 10 DNA bases. In some examples, the one or more matching DNA bases may be at the 3′ end of the cleavage site.

In some examples, the one or more mismatching DNA base may comprise 1 DNA base, 2 DNA bases, 3 DNA bases, 4 DNA bases, 5 DNA bases, 6 DNA bases, 7 DNA bases, 8 DNA bases, 9 DNA bases, or 10 DNA bases. In some examples, the one or more mismatching DNA base may be at the 3′ end of the matching DNA bases. In some examples, the primer may comprise one mismatching DNA base at the 3′ end of the primer.

In some examples, the primer and/or probe may comprise one or more blocking group. In some examples, the primer and/or probe may comprise 1, 2, 3, 4, 5, or more blocking groups. In some examples, the primer and/or probe may comprise 1 blocking group. In some examples, the primer and/or probe may comprise 2 blocking groups.

As disclosed herein, the blocking group may be a chemical moiety that is bound to the primer or other oligonucleotide such that an amplification reaction does not occur. For example, primer extension and/or DNA ligation does not occur. Once the blocking group is removed from the primer or other oligonucleotide, the oligonucleotide is capable of participating in the assay for which it was designed (e.g. PCR, ligation, sequencing, etc). Thus, the blocking group can be any chemical moiety that inhibits recognition by a polymerase or DNA ligase. The blocking group may be incorporated into the cleavage domain but is generally located on either the 5′- or 3′-side of the cleavage domain. In some examples, the blocking group is on the 3′ end of the oligonucleotide. The blocking group can be comprised of more than one chemical moiety. In the present invention the “blocking group” is typically removed after hybridization of the oligonucleotide to its target sequence. In some examples, the blocking group may be a C3 spacer (a phosphoramidite,

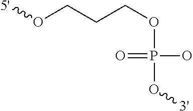

(for incorporation at 5′ end or internally), or

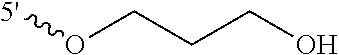

(for incorporation at 3′ end)), a hexanediol (a six carbon glycol spacer or

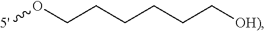

a 1′2′-dideoxiribose (dSpacer or

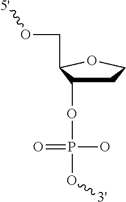

(for incorporation at 5′ end, internally, or 3′ end)), a PC Spacer (

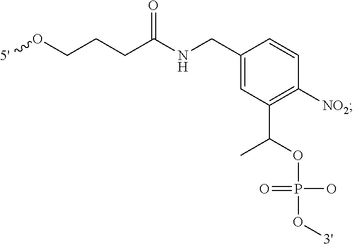

(for incorporation at the 5′ end or internally), a Spacer 9 (a thriethylene glycol spacer

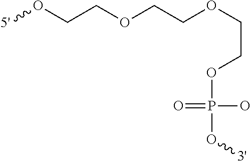

for incorporation at the 5′ end, internally, or 3′ end)), a Spacer 18 (18-atom hexa-ethyleneglycol spacer, or

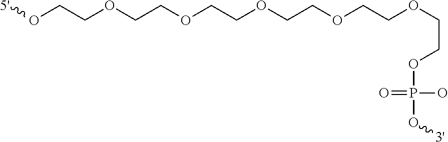

(for incorporation at the 5′ end, internally, or 3′ end)).

In some examples, the blocking group may be provided at the 3′ end of the primer. In some examples, the blocking group may be provided at the 3′ end of a mismatching DNA. In some examples, the blocking group may be provided within the one or more matching DNA bases and at the 5′ end of the mismatching DNA base. In some examples, where high fidelity of template amplification is desired, the blocking group is provided within the one or more matching DNA bases at the 5′ end of the mismatching DNA bases.

In some examples, the primer and/or probe may comprise formula (I):

Dₙ₁-Rₙ₂-Dₙ₃-Mₙ₄-Dₙ₃-X (I)
wherein
D is a DNA base that match target nucleic acid,
R is an RNA base that matches target,
M is a DNA base that is a mismatch to target nucleic acid,
X is a blocking group,
n1 is an integer representing the number of bases of between 1 to 20,
n2 is an integer representing the number of bases of between 1 to 5, and
n3 is an integer representing the number of bases of between 0 to 5.

In some examples, the primer and/or probe may comprise, in order from 5′ to 3′, a functional primer, a cleavage site, one or more matching DNA bases, one or more mismatch DNA base, and one or more blocking group.

In some examples, the primer and/or probe may comprise, in order from 5′ to 3′, a functional primer, a cleavage site, one or more matching DNA bases, one or more blocking group, one or more matching DNA bases, and one or more mismatching DNA.

In some examples, the primer comprising one or more RNA bases is an rhPCR primer (i.e. an RNase-dependent PCR primers), optionally the RNase-dependent PCR primers is an RNase H-dependent PCR primers. When the primer is an RNase H-dependent PCR primers, which is described in US 2015/225782 A1, the content of which is incorporated herein by reference.

The inventors of the present disclosure found that the combination of amplification of the target nucleic acid in surfactant (i.e. emulsion based PCR) with the oligonucleotide having cleavage site (i.e. rhPCR primers) advantageously increases the specificity of the amplification method.

In some examples, the present disclosure also includes the use of a probe. In some examples, the probe may be an oligonucleotide attached/conjugated to a detectable agent (such as a fluorophore and/or quencher). In some examples, the probe may be an oligonucleotide attached/conjugated to a detectable agent (such as a fluorescent label and/or quencher) and a groove binder. In some examples, the probe may comprise a nucleic acid binding reagent (such as SYBR® Green dye).

In some examples, the control nucleic acid is added to the sample at a constant amount to thereby normalizes of the amplification efficiency across a plurality of samples, optionally the control nucleic acid is added to the sample at about 10² to 10¹⁰ copies.

In some examples, the control nucleic acid (i.e. spike-in controls) does not compete or interfere with the amplification of the target nucleic acid. In some examples, the controls have low sequence homology to the target nucleic acid (for example it has low sequence homology to any human genes). In some examples, the control nucleic acid has a different sequence from the target nucleic acid. In some examples, the control nucleic acid is nucleic acid that cannot be found in the sample (i.e. exogenous from the sample) and/or is not a housekeeping gene. In some examples, the control nucleic acid is included in greater abundance than the target nucleic acid. The addition of a control nucleic acid allows for normalization of the technical amplification efficiency across samples. The control nucleic acid also advantageously normalizes for any unintended variation in the experiment.

In some examples, the control nucleic acid may be a DNA and/or RNA. In some examples, the control nucleic acid may be substantially no or very low sequence homology or substantially different from human gene. In some examples, the control nucleic acid may be luciferase. In some examples, the control nucleic acid may be luciferase RNA. As illustrated in the Experimental Section, the methods as disclosed herein may include the usage of luciferase RNA as a spiked-in for normalising PCR efficiency.

Without wishing to be bound by theory, it is believed that the method as disclosed herein may leverage on the “CoT effect” that increases the sensitivity of a method with minimal loss in linearity when used in quantitative methods. As used herein, the “CoT effect” refers to an amplification method where the presence of greater abundance of a particular nucleic acid results in a systemic bias against the more abundant of the two PCR products (one being an abundant nucleic acid (may be an internal control or an endogenous nucleic acid present in abundance in the sample) and the other being the target nucleic acid). The slowdown in amplification of abundant products allows the target nucleic acid/target of interest (which may be present in less quantity) to become more visible in the fingerprint. It is believed that the increase visibility of the target nucleic acid/target of interest (which may be present in lesser quantities) allow the target of interest (such as rarer cDNAs) to be sampled more efficiently. In another word, CoT PCR enable selective amplification of low concentration DNA resulting in increase of sensitivity for downstream applications.

In some examples, the methods as disclosed herein may comprise CoT PCR. In some examples, the amplification step in the method as described herein includes interposing an annealing step between denaturation and priming. Without wishing to be bound by theory, since the amount of re-annealing depends on the product of the initial concentration and time, CoT, the more abundant a sequence the greater will be the extent of its conversion to the double-stranded form. These hybrids will then fail to be copied in the polymerisation step of the cycle. Thus, as each sequence reaches the threshold concentration at which considerable re-annealing occurs it ceases to be exponentially amplified, and in this way, all sequences will eventually have the same concentration. In some examples, the CoT PCR maybe as described by Brenner S. and Jones DSC, 1972 (Wellcome collection, which can be accessed here: https://wellcomecollection.org/works/h39jksrt/items, the content of which is incorporated herein).

In some examples, the control nucleic acid is a nucleic acid that may be added to the method as disclosed herein in a fixed amount (or a constant amount in all samples). In some examples, the control nucleic acid is provided at a concentration that is higher than the predicted concentration of the target of interest (or target nucleic acid).

In some examples, the amount of control nucleic acid added to the sample is about 10² to 10¹⁰ copies. In some examples, the amount of control nucleic acid maybe about 100 copies, 10³ copies, 10⁴ copies, 10⁵ copies, 10⁶ copies, 10⁷ copies, 10⁸ copies, 10⁹ copies, or 10¹⁰ copies. In some examples, the amount of control nucleic acid may be about 100 to 10⁹ copies, or about 100 to 10⁸ copies, or about 100 to 10⁷ copies, or about 100 to 10⁶ copies, or about 100 to 10⁵ copies. In some examples, the amount of control nucleic acid is about 10⁵ copies.

In some examples, the method may further comprise the detection of a second control nucleic acid that is present endogenously in the sample. For example, the second control nucleic acid may be a housekeeping gene. In some examples, the second control nucleic acid may include, but is not limited to, actin beta (ACTB), glyceraldehyde 3-phosphate dehydrogenase (GAPDH), ribosomal protein S18 (RPS18), ubiquitin C (UBC), beta-2 microglobulin (B2M), glucuronidase beta (GUSB), hypoxanthine-guanine phosphoribosyltransferase (HPRT), phosphoglycerate kinase 1 (PGK1), peptidylprolyl isomerase A (PPIA), TATA box binding protein (TBP), transferrin receptor (TFRC), tyrosine 3-monoxygenase/tryptophan 5-monooxygenase activation protein zeta (YWHAZ), tubulin, heat shock protein 90 (HSP90), hypoxanthine guanine phosphoribosyl transferase (HPRT), succinate dehydrogenase complex, subunit A (SDHA), mitochondrially encoded 12S ribosomal RNA (mtRNR1), mitochondrially encoded 16S RNA (mtRNR2), and the like. The detection of the second control nucleic acid allows for the normalization of extraction efficiency across samples.

In some examples, the method further comprises analysing data by normalizing raw values (such as Ct value) to the levels of control nucleic acids (such as housekeeping gene, spiked in luciferase RNA).

In some examples, the method comprises contacting the target nucleic acid with an annealing reagent comprising a primer of the target nucleic acid, a control nucleic acid, and an annealing mixture (including deoxyribonucleotide triphosphate (dNTP) mixes). In some examples, where the nucleic acid is an RNA, the method comprises contacting the target nucleic acid with an annealing reagent comprising a reverse primer of the target nucleic acid, a control nucleic acid, and an annealing mixture (including deoxyribonucleotide triphosphate (dNTP) mixes).

In some examples, the annealing step precede the reverse transcription and amplification cycles.

In some examples, the method further comprises subjecting the target nucleic acid to reverse transcription. In some examples, the method further comprises a reverse transcription of the target nucleic acid after annealing step.

In some examples, the method comprises contacting the target nucleic acid with a reverse transcription agent comprising a reverse transcriptase, and a reverse transcriptase mixture (including DTT).

In some examples, the method further comprises inactivation of the reverse transcriptase.

In some examples, the method further comprises a step of quantifying the amount of target nucleic acid present in the sample and/or sequencing the target nucleic acid in the sample.

In some examples, the method of the present disclosure may be adaptable to include processing where amplified cDNA exhibits compatibility for downstream further processing. This is because the method of the present disclosure advantageously provides an adaptable end point where amplified cDNA exhibit compatibility for downstream quantification using methods known in the art. For example, the cDNA as amplified by the method as disclosed herein may be used in further steps of quantifying the amount of target nucleic acid by performing quantitative real-time PCR, next generation sequencing, UV absorbance with spectrophotometer, fluorescence dyes, agarose gel electrophoresis, microfluidic capillary electrophoresis, diphenylamine method, droplet digital PCR, and the like.

Diverse RNA transcripts are widely detected to be circulating within the human plasma. The notion of using these circulating cell free RNA (cfRNA) as potential biomarkers has recently emerged from comprehensive assessments using high throughout sequencing technologies that led to the identification of tissue-specific cfRNA changes that correlate to pathological conditions such as cancer and metabolic diseases. Follow up work by the inventors have also shown that tissue specific cfRNA provides a non-invasive window for studying hard to reach tissues under different biological conditions.

Quantification of tissue-specific cfRNA is expected to vary based on the tissue of origin and the biological state of the cells when releasing cfRNA via apoptosis. However, the lack of effective and sensitive molecular tools to amplify and quantitate RNA biases against low abundance tissue specific cfRNA of interest. This in turn limits the widespread use of cfRNA as biomarkers. To address this, the present disclosure discloses a molecular protocol that overcome this by combining emulsion-based PCR together with specifically designed rhprimers that pre-amplifies tissue specific cfRNA for downstream quantitation with qPCR, or next generation sequencing.

Therefore, in some examples, the nucleic acid is a cell free nucleic acid, optionally a circulating cell free nucleic acid. In some examples, the cell free nucleic acid is a cell free DNA and/or a cell free RNA. In some examples, the nucleic acid is a circulating cell free RNA. In some examples, the cell free nucleic acid is an isolated cell free nucleic acid.

In some examples, when the nucleic acid is an RNA, the method comprises annealing the target nucleic acid is in the presence of a reverse primer of the target nucleic acid and the control nucleic acid, subjecting the annealed sample to reverse transcription, and subjecting the target nucleic acid to one or more amplification step in the presence of a mixture comprising a surfactant and a primer comprising one or more RNA base and a cleavable 3′ end.

In some examples, the reverse primers of the target nucleic acid may comprise about 10 to 40 bases, or 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 35 bases, or 40 bases. In some examples, the reverse primers of the target nucleic acid may be about 15-18 bases, or about 16 bases.

In some examples, the method further comprises the extraction of the nucleic acid from a sample.

In some examples, the sample may include any items that may contain nucleic acid of interest. For example, the items may be a surface of an equipment, a laboratory bench, a public surface (such as, but not limited to, surface on an elevator/lift/doorknobs/toilet, surface on a public transport, surface of airport areas, surface of school areas, surface of shopping mall or supermarket areas, surface of restaurants/hawkers/cafes, and the like), frequently touched surfaces adjacent to patients in hospitals/clinics (such as, but not limited to, areas adjacent to or at the hospital bed, hospital/clinic waiting areas, quarantine rooms and the like).

In some examples, the sample may be a biological sample. In some examples, the nucleic acid is obtained from a biological sample.

In some examples, the samples may be obtained at different time points of the disease state. For examples, the disease state may include pre-surgery, peri-operative period, immediately after surgery, short term post-surgery, long-term post-surgery, antibody positive state, recurrent or persistent cancer, and the like.

The presence or absence of a target nucleic acid can be measured quantitatively or qualitatively. Target nucleic acid can come in a variety of different forms including, for example, simple or complex mixtures, or in substantially purified forms. For example, a target nucleic acid can be a part of a sample that contains other components or can be the sole or major component of the sample. Therefore, a target nucleic acid can be a component of the circulatory system (such as blood, serum, plasma, or combinations thereof), a whole cell or tissue, a cell or tissue extract, a fractionated lysate thereof or a substantially purified molecule. In some examples, the target nucleic acid is a region of interest in a cell free DNA and/or RNA. In some examples, the target nucleic acid is a region of interest in a cell free RNA. In some examples, the target nucleic acid is a region of interest in a circulating cell free RNA.

In some examples, the method detects the presence and/or absence of any one of the following interest, such as, but not limited to, a pathogen, a disease, a cancer, a genetic defect, and the like. For example, pathogens may be a bacterial pathogen, a viral pathogen, a fungal pathogen, or a parasite.

Examples of a bacterial pathogen may include, but is not limited to, Escherichia coli, Mycobacteria spp, Salmonella spp, Staphylococcus spp, Clostridium difficile, Listeria monocytogenes, Group B streptococci, vancomycin-resistant enterococci (VRE), and the like.

Examples of a viral pathogen may include, but is not limited to, Human papillomavirus, Rhinovirus, Human cytomegalovirus in HIV-1 positive patient, Hepatitis virus, Coronavirus (CoV), severe acute respiratory syndrome (SARS), monkey pox virus and the like.

Examples of a fungal pathogen may include, but is not limited to, Botrytis cinerea, Pseudomonas syringae, Fusarium oxysporum and the like.

Examples of a parasite may include, but is not limited to, Leishmania parasites, Giardia, Cryptosporidum, Entamoeba and the like.

In some examples, the disease may be a metabolic disorder, such as, but is not limited to, hypothyroidism, hyperthyroidism, diabetes, mitochondrial disorders, phenylketonuria (PKU), and the like.

In some examples, the cancer may include, but is not limited to, thyroid cancer, pancreatic cancer, breast cancer, colon cancer, lung cancer, liver cancer, skin cancer, and the like.

In some examples, the genetic defects may include, but is not limited to, a prenatal genetic defect, Cystic fibrosis, and the like. In some examples, the prenatal genetic defect may include, but is not limited to, Down syndrome (Trisomy 21), Turner Syndrome, Edwards' syndrome, and the like.

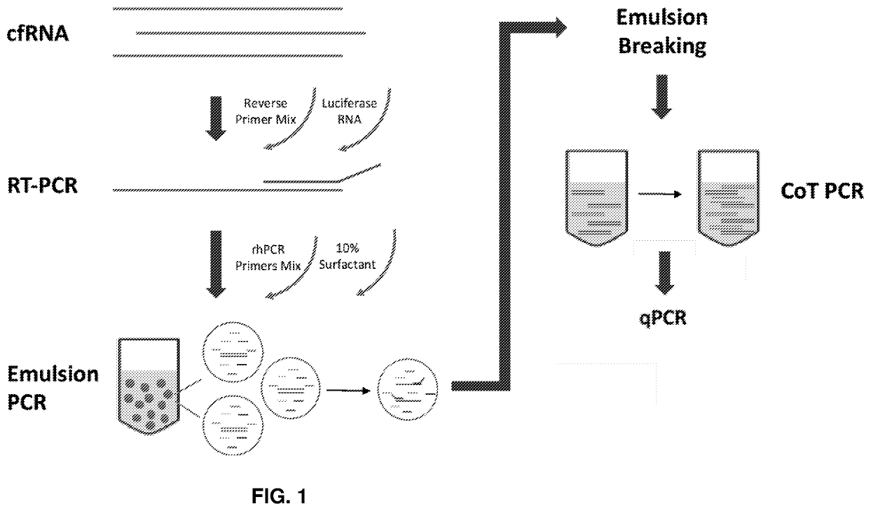

The present invention can advantageously be performed as a “one-pot amplification” process. The one-pot amplification made possible by the use of the emulsion PCR and the spiking of the sample with a control nucleic acid. As illustrated in FIG. 1, the methods as disclosed herein can be performed in a single closed tube from annealing step to reverse transcription and amplification step.

In some examples, the method is a real time amplification method.

In some examples, the method as disclosed herein may be performed on samples drawn at multiple time points. In some examples, the samples may be drawn/obtained from the subject at one or more, 2 or more, 3 or more, 4 or more, 5 or more, 6 or more, 7 or more, 8 or more, 9 or more, or 10 or more time points. In some examples, the samples may be drawn/obtained from the subject at 1 to 10 time points, or at least at 1 time point, or at least at 2 time points, or at least at 3 time points, or at least at 4 time points, or at least at 5 time points, or at least at 6 time points, or at least at 7 time points, or at least at 8 time points, or at least at 9 time points, or at least at 10 time points. In some examples, the samples may be drawn/obtained from the subject at least 5 time points. For example, the 5 time points may include pre-surgery, short-term post-surgery, long-term post-surgery, and the like.

In another aspect, there is provided a nucleic acid amplification mixture comprising a first mixture comprising: a control nucleic acid, and a second mixture comprising: a surfactant, and an oligonucleotide primer and/or probe capable of hybridizing with a target nucleic acid, wherein the oligonucleotide primer and/or probe comprises a cleavage site and a cleavable 3′ end.

In some examples, the second mixture further comprises amplification reagents.

In some examples, wherein the amplification agent comprises detectable primers and/or probes.

In some examples, there is provided a kit comprising the reagents and/or mixtures used in the methods as disclosed herein.

In some examples, the target nucleic acid may be present in the sample in minute amount or in low quantity. In some examples, the target nucleic acid may not be present in abundance.

In some examples, the amount of sample nucleic acid may be about 1 μL to about 100 μL. In some examples, the amount of cfRNA in the sample may be about 1 μL to 90 μL, or about 5 μL to about 80 μL, or about 10 μL to about 50 μL. In some examples, the amount of cfRNA in the sample may be no more than 50 μL, no more than 40 μL, no more than 30 μL, no more than 20 μL, no more than 19 μL, no more than 18 μL, no more than 17 μL, no more than 16 μL, no more than 15 μL, no more than 14 μL, no more than 13 μL, no more than 12 μL, no more than 11 μL, no more than 10 μL, and the like.

In some examples, the amount of sample nucleic acid may be about 500 picogram (pg) to about 1000 μg. In some examples, the amount of sample nucleic acid may be about 500 pg, may be about 550 pg, may be about 600 pg, may be about 650 pg, may be about 700 pg, may be about 750 pg, may be about 800 pg, may be about 850 pg, may be about 900 pg, may be about 950 pg, may be about 1000 pg, may be about 1050 pg, may be about 1100 pg, may be about 1150 pg, may be about 1200 pg, may be about 1300 pg, may be about 1400 pg, may be about 1500 pg, may be about 2000 pg, may be about 3000 pg, may be about 4000 pg, may be about 5000 pg, may be about 6000 pg, may be about 7000 pg, may be about 8000 pg, may be about 9000 pg, may be about 10,000 pg, may be about 15,000 pg, may be about 20,000 pg, may be about 25,000 pg, may be about 30,000 pg, may be about 35,000 pg, may be about 40,000 pg, may be about 50,000 pg, may be about 100,000 pg, may be about 0.01 μg, may be about 0.02 μg, may be about 0.03 μg, may be about 0.04 μg, may be about 0.05 μg, may be about 0.06 μg, may be about 0.07 μg, may be about 0.08 μg, may be about 0.09 μg, may be about 0.1 μg, may be about 0.2 μg, may be about 0.3 μg, may be about 0.4 μg, may be about 0.5 μg, may be about 0.6 μg, may be about 0.7 μg, may be about 0.8 μg, may be about 0.9 μg, or may be about 1 μg. In some examples, the amount of sample nucleic acid may be no more than 2 μg, 3 μg, 4 μg, 5 μg, 6 μg, 7 μg, 8 μg, 9 μg, 10 μg, 11 μg, 12 μg, 13 μg, 14 μg, 15 μg, 20 μg, 30 μg, 40 μg, 50 μg, 100 μg, 150 μg, 200 μg, 300 μg, 400 μg, or 500 μg, or 2 to 1000 μg.

In another aspect, there is provided a method of detecting and/or determining the presence and/or the amount of a target nucleic acid comprising annealing the target nucleic acid in the presence of a control nucleic acid, and subjecting the target nucleic acid to one or more amplification step in the presence of a mixture comprising a surfactant and an oligonucleotide capable of hybridizing to the target nucleic acid comprising one or more RNA base and a cleavable 3′ end.

Also disclosed are methods of quantifying tissue/organ health in disease states. For example, the methods as disclosed herein may be applied to metabolic disorders and cancer surveillance.

As such, the present disclosure provides sensitive and multiplex methods for targeted amplification and/or quantification of low amounts of naturally occurring tissue specific RNA extracted from plasma.

In some examples, the method may comprise: (a) providing a reaction mixture comprising (i) rhPCR primers (e.g. an oligonucleotide primer having a cleavage domain positioned 5′ of a blocking group and 3′ of a position of variant, the blocking group linked at or near the end of the 3′-end of the oligonucleotide primer wherein the blocking group prevents primer extension and/or inhibits the oligonucleotide primer from serving as a template for DNA synthesis), (ii) a control RNA, (b) subjecting the reaction mixture to reverse transcription conditions, (c) contacting the resulting mixture from step (b) with a surfactant and an amplification mixture, (d) subjecting the mixture from step (c) to amplification conditions sufficient to result in the amplification of the nucleic acid.

In some examples, there is provided a method of amplification of a target nucleic acid, the method comprising annealing the target nucleic acid in the presence of a reverse primer of the target nucleic acid and a control nucleic acid, subjecting the target nucleic acid to reverse transcription, and subjecting the target nucleic acid to one or more amplification step in the presence of a mixture comprising a surfactant and a primer comprising one or more RNA base and a cleavable 3′ end.

### Experimental Section

### Material & Methods

### Tissue Specific Gene Selection Using Human Protein Atlas

Genes that are specific to the pancreas were selected based on the Human Protein Atlas (https://www.proteinatlas.org/). Primers were designed and validated to amplify cross-exons region by using UCSC Genome Browser in-silico PCR tool. (https://genome.ucsc.edu/).

### Plasma Sample Collection

Blood samples were acquired though the National University of Hospital following DSRB approval. Whole blood was subjected to two-stage centrifugation (1600×G and 16000×G) within a day after collection and the plasma collected were stored in 1 mL aliquots at −80° C. before RNA extraction.

### Primer Design

Rnase H-dependent PCR (rhPCR) primers were designed according to the Integrated DNA Technologies Gen1 design. The primer consists of five different parts starting at the 5′ end with the final functional primer (comprising of more than 10 DNA bases that matches the template), the cleavage site (single RNA residue), four matching DNA bases, one mismatch DNA base, and lastly the blocking group (C3 spacer) at the 3′ end.

### Selection of Pancreatic-Specific Targets for Measurements in the Plasma

A data-driven approach leveraging on pancreatic-specific targets derived from the Human Protein Atlas was adopted to measure tissue-specific RNA within the plasma. Genes that fall into the category of “Tissue-enriched genes” in pancreatic tissues were selected. Highly expressed and biologically significant genes were selected for the panel. 14 pancreatic-specific genes were targeted which include KLK1, CTRB1, ERP27, IAPP, PRSS1, CELA3A, PLA2G1B, PNLIP, CUZD1, CPB1, CPA1, CLPS, INS, and GCG.

### Circulating RNA Extraction

Consistent and reliable blood collection protocols are critical to maintain the integrity of circulating nucleic acid assays. Blood was spun down twice (once at 2000×g and a high speed second spin at 16000×g) to separate blood cells. The resulting plasma is collected for downstream extraction. Blood was processed within 6 hours to prevent cell lysis and reduce degradation of RNA.

cfmRNA was extracted from 1 mL of plasma using a Plasma/Serum Circulating and an Exosomal RNA Purification Kit (Norgen, Cat no. 42800). The residual DNA in the cfmRNA was digested using an RNase-Free DNase I Kit (Norgen, Cat no. 25720). Extracted cfmRNA was purified using an RNA Clean & Concentrator™-5 (Zymo, Cat no. ZYR.R1016), yielding 24 μL of cfmRNA per sample.

### Reverse Transcription & Emulsion Based Targeted Pre-Amplification

10 μL of extracted cfmRNA was annealed with 0.4 μM of reverse primers mix in the presence of 10,000 copies of Luciferase control RNA (Promega, Cat no. L4561) and 2 mM of dNTPs at 65° C. for 5 minutes. Reverse transcription of cfmRNA was performed using Superscript™ III Reverse Transcriptase (Invitrogen, Cat no. 18080044) at 25° C. for 5 minutes, 50° C. for 50 minutes, and enzyme inactivation at 95° C. for 3 minutes.

cDNA from reverse transcription was added to the PCR mixture of Platinum™ Taq DNA Polymerase (Invitrogen, Cat no. 10966) with 0.5 μM of rh PCR primers mix and 26 mU of RNase H2 enzyme. Emulsion was generated by adding 3 parts of 10% 008-FluoroSurfactant (RAN Biotechnologies) in 3M Fluorinert™ Engineered Fluid (3M, Cat no. FC-40) to 1 part of PCR reaction mixture. The mixture was vortexed until it became cloudy and uniform. Thermocycling started with enzyme activation at 94° C. for 2 minutes, followed by 20 cycles of denaturation (94° C., 15 seconds), annealing (55° C., 30 seconds), and extension (68° C., 1 minute). Reaction recovered from emulsion PCR was topped up with the same amount of polymerase and RNase H2 used in emulsion PCR. Second thermocycling reaction started with enzyme activation at 94° C. for 2 minutes, followed by 20 cycles of denaturation (94° C., 15 seconds), hybridization (78° C., 10 minutes), annealing (55° C., 30 seconds), and extension (68° C., 1 minute).

### Emulsion Breaking

Reaction was frozen at −80° C. for 1 hour before thawing at room temperature. The top fraction containing the reaction mix was transferred to a fresh tube. Fraction that was recovered was topped up with the same amount of Platinum™ Taq polymerase and RNase H2 enzyme used in the previous PCR reaction. Emulsion breaking may or may not be included in the Emulsion PCR step.

### Quantification of Pre-Amplified Gene Targets Using qPCR

To monitor the expression of the targeted cfmRNA across the different time point, qPCR was performed for 60 cycles with Maxima SYBR Green/ROX qPCR Master Mix (ThermoFisher Scientific, Cat no. K0221).

### Spike-In Controls and Quality Controls Assays for Ensuring Consistent Comparisons Across Patient Samples

There were two types of quality control. One for extraction efficiency and another for technical amplification efficiency. Extraction efficiency was normalized across samples using the levels of housekeeping gene: ACTB, GAPDH, and RPS18. Technical amplification efficiency was normalized across samples using a constant amount of spiked in Luciferase RNA that is not normally found in normal plasma (exogenous RNA). 10⁵ copies of Luciferase Control RNA (Promega, Cat no. L4561) was spiked in with 10 μL of the extracted cfmRNA at the reverse transcription step. It was used as a control to normalize for any unintended variation in the experiment.

### Data Analysis

Melting temperatures of the circulating pancreatic transcripts are compared to the reference melting temperature of the amplicons obtained from using commercially obtained RNA (Takara Bio) extracted from pancreatic tissues. Mismatch in amplicon melting temperature indicates a wrong amplification product and the corresponding Ct value will be set to 60 which is the maximum number of cycles.

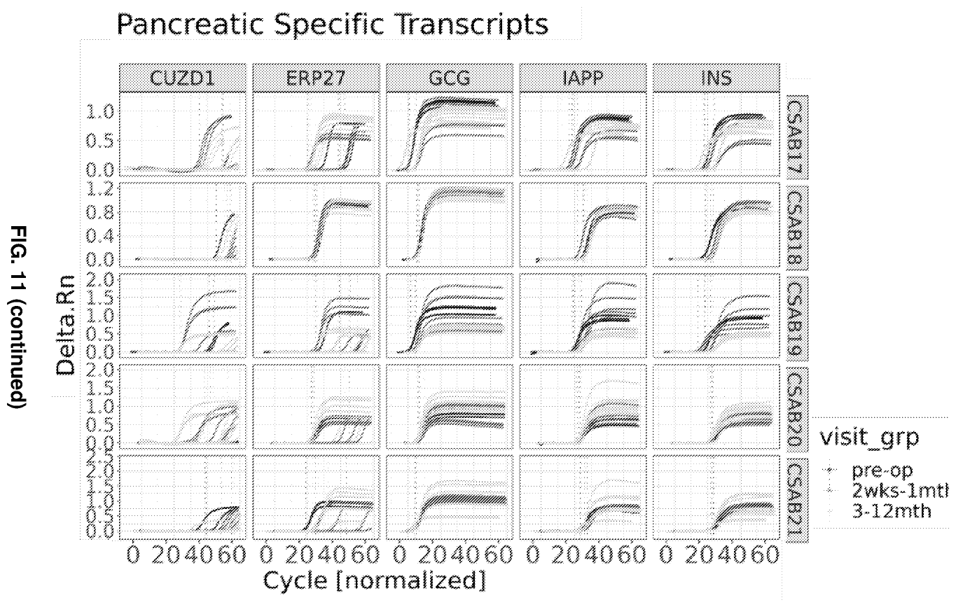

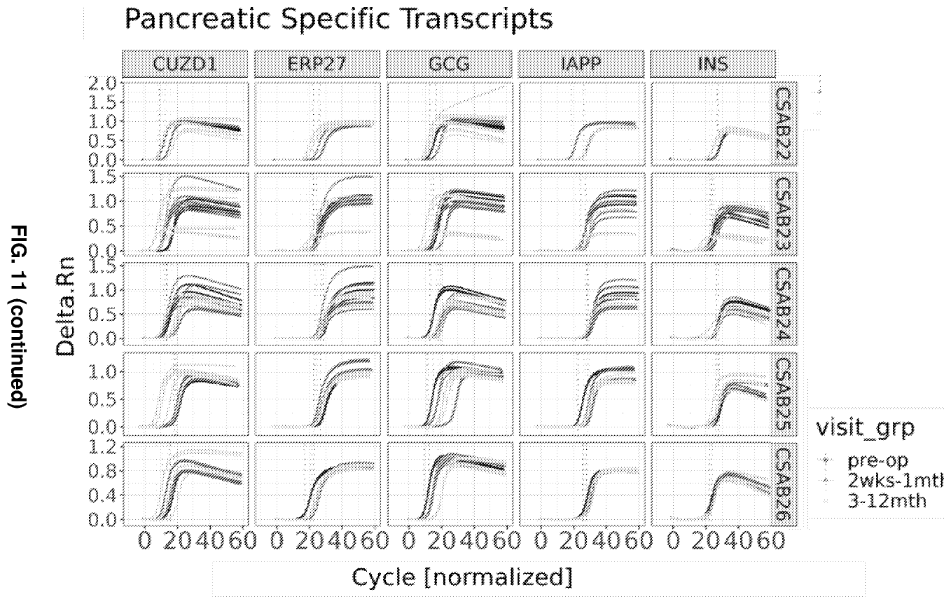

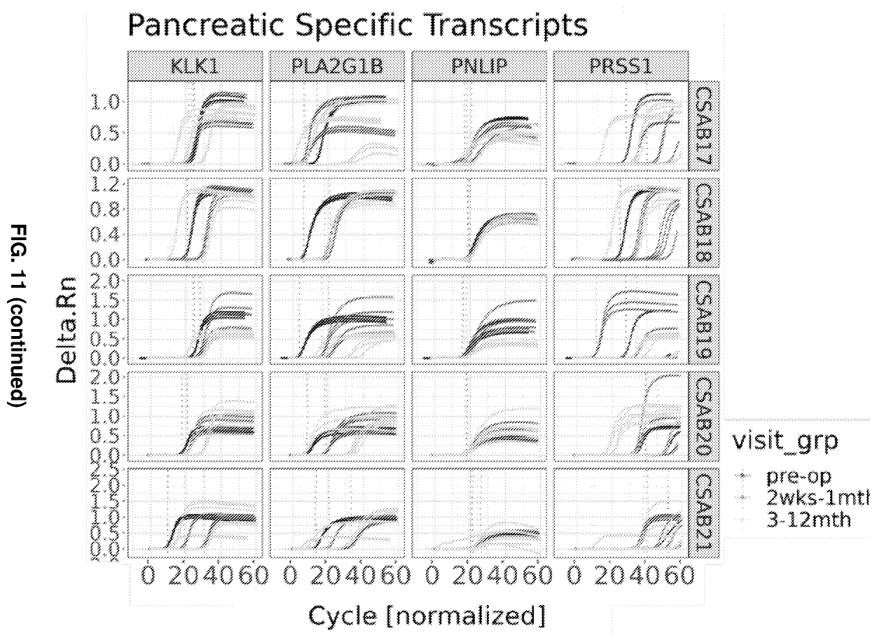

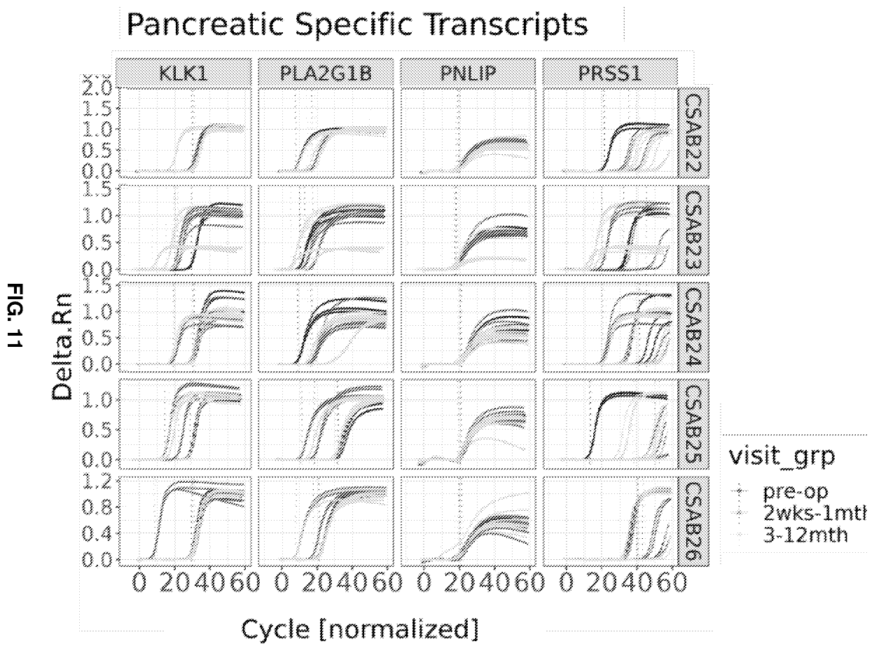

Ct values of the transcripts then undergo two normalization steps. The first normalization step is to normalize to the Ct value of the spiked in luciferase which will account for technical variation during the PCR steps across different samples. The second normalization step uses the geometric mean of the housekeeping genes and is meant to account for variations in the amounts of extracted RNA. Paired wilcox test is then performed on the normalized Ct values comparing the transcript levels pre-surgery and post-surgery (short and long term). All melt curves and amplification plots are included as FIG. 10 and FIG. 11.

Data was analyzed using normalized Ct values with respect to the housekeeping genes and are corrected for amplification efficiency using the amounts of spiked in luciferase RNA. The inventors of the present disclosure normalized the raw Ct values with Luciferase control RNA and housekeeping genes, and verified the amplified product by referring to the melt curve. For an undetermined Ct/incorrect product, Ct value of 60 was assigned due to the 60 cycles of qPCR. +The final Ct value was then deducted from 60 to allow easy visualization of the changes in RNA expression.

### Results

### Validation of the Cot-Emulsion PCR Molecular Technology Protocol Using Artificial Spike in Controls

Luciferase RNA (LUC), which is not found in normal plasma RNA, is used as a spiked-in control to mimic the presence of low level of circulating RNA of interest across different applications. A range of LUC copies are used: 2¹⁶ [65536 molecules], 2¹⁸ [262144 molecules], 2²² [4,194,304 molecules] are spiked into RNA extracted from 1 ml of human plasma. These range of spiked-ins are used to illustrate the range of operability as well as scalability of the protocol.

These spiked in samples are put through 2 different versions of the molecular protocol. The first protocol comprises all the major steps including CoT amplification, to validate that the molecular technology is detecting the spike in LUC molecules. The second protocol has the CoT amplification process removed, to validate and illustrate the impact of CoT amplification in improving the Ct measurements and sensitivity of detection. In addition to LUC, housekeeping genes [RPS18, ACTB] are also used as positive controls for the platform.

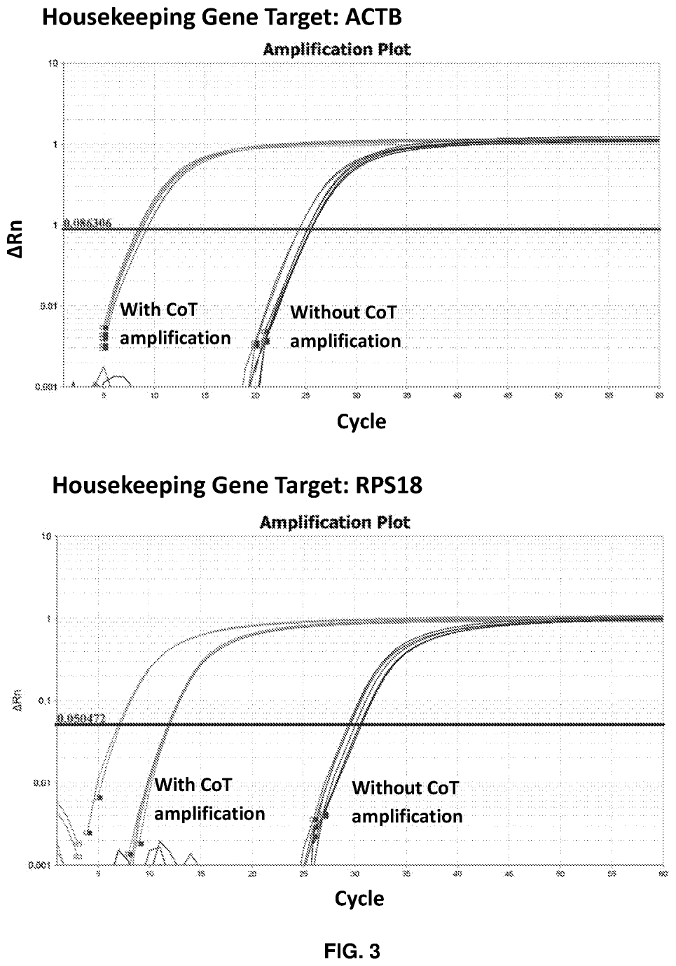

The inventors of the present disclosure show that the current protocol amplifies the target spiked in LUC and the quantified Ct cycles scales with the input range of molecules. In addition, CoT amplification significantly improves the sensitivity of the protocol by decreasing the Ct cycles. (FIG. 3 to FIG. 6).

### Multiplex Amplification of Pancreatic Specific cfmRNA from the Plasma

Circulating cell free mRNA (cfmRNA) has been shown to contain transcripts that are derived from multiple organs and can be used to capture tissue-specific transcriptional change. Many of these cfmRNA transcriptome sequencing studies have also shown that tissue-specific circulating RNA can be present at very low levels in the plasma. To address the potentially low levels of pancreatic specific RNA in plasma, the inventors of the present disclosure chose a targeted approach and integrated three highly sensitive multiplex PCR strategies (RNaseH2-dependent PCR, Emulsion PCR & CoT PCR) from other studies known in the art into a single workflow.

An overview of the circulatory RNA amplification workflow is presented in FIG. 1. The first step is deploying multiplexed pre-amplification using RNaseH2-dependent PCR (rhPCR) after the initial reverse transcription of cfmRNA. Exon-spanning rhPCR primers (rhPrimers) were designed for each of the 14 pancreatic gene targets. Each rhPrimers contains a single RNA base and are used in conjunction with thermostable RNase H2 enzyme to perform rhPCR. When the rhPrimer anneals to the intended pancreatic specific target cDNA, a substrate for RNaseH2 is formed at the site where the single RNA residue resides in the primer. When there is perfect complementarity, RNaseH2 cleaves the RNA site, removing the blocking group from the primer to facilitate DNA synthesis. Imperfect complementarity does not result in this unblocking reaction. This results in improved specificity of the priming event and allows for targeted multiplex cDNA amplification.

To further reduce the formation of any unwanted chimeric products, the rhPCR step is performed using emulsion-based PCR technique where aqueous rhPCR mix is compartmentalized into an oil-water emulsion before thermocycling. The resulting amplicons are subsequently recovered by freezing the mix at −80° C. which breaks the emulsion. Recovered amplicons are then put through CoT PCR enrichment that preferentially amplifies rare amplicons over abundant ones by taking advantage of the CoT effect⁰.

Additional housekeeping genes (ACTB, GAPDH, RPS18) and Luciferase (LUC) gene are included as quality control targets in the assay to ensure that results are comparable across plasma samples after amplification. Quantification of housekeeping genes are used for normalization of cfmRNA extraction efficiency. Moreover, each of the extracted cfmRNA samples are spiked with a 10⁵ molecules of commercially obtained Luciferase Control RNA to normalize technical variability arising from the amplification process.

### Screening for Temporal Changes in Circulating Pancreatic cfmRNA

Plasma was collected from each patient across at least five different visits that spans three main phases: pre-surgery, short-term post-surgery (two weeks to one month) and long-term post-surgery (three to 12 months). cfmRNA extracted from 1 ml of plasma was quantified for pancreatic specific transcripts using the methods described. The inventors of the present disclosure found that one of the pancreatic transcripts CTRB1 (Chymotrypsinogen B1) was not amplified from the plasma and hence was not detected using qPCR. Of the remaining 13 transcripts that were amplified, the inventors of the present disclosure performed the Wilcoxon paired test comparing the qPCR Ct values of each pancreatic specific transcript pairwise across different visits i.e. (1) pre-surgery vs short-term post-surgery, (2) pre-surgery vs long-term post-surgery as shown in Error! Reference source not found.7. Although temporal variations were observed amongst the remaining 13 amplifiable transcripts within individual patients, changes in GCG level is the most statistically consistent (p-value: 0.037) when comparing pre-surgery vs short-term post-surgery levels. Higher Ct levels are observed for circulating GCG transcripts during the short-term post-surgery period which translates to a decrease in amounts of circulating GCG transcripts during the period immediately post-surgery.

### GCG cfmRNA Levels Correlates with Common Clinical Biomarkers Used to Monitor Bariatric Surgery

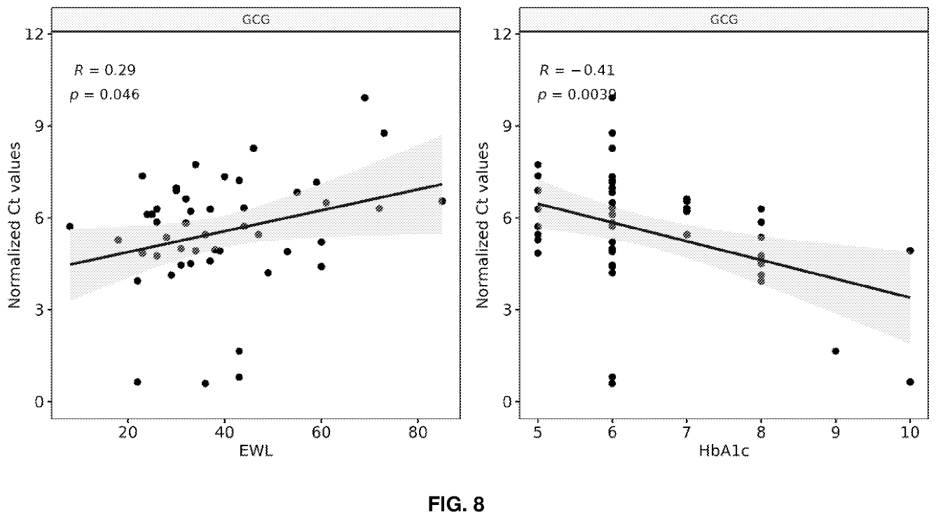

The inventors of the present disclosure correlated Ct measurements of GCG cfmRNA against clinical biomarkers collected routinely to track the outcome of the bariatric surgery. GCG cfmRNA Ct measurements correlates positively (R: 0.29, p-value: 0.046) with the percentage of excess weight loss (% EWL) and exhibit an inverse correlation (R: −0.41, p-value: 0.0039) with glycated haemoglobin (HbA1c) levels as shown in FIG. 8. % EWL is a common metric for quantifying weight loss due to bariatric surgery whereas HbA1c is typically used to quantify metabolic outcomes such as the extent of diabetes. Here the inventors of the present disclosure noticed that levels of GCG cfmRNA captures both weight loss and metabolic outcome metrics and could potentially serve as an additional metric for monitoring surgical outcomes.

GCG gene expression is pivotal for metabolic regulation, encoding for multiple peptides including glucagon, glucagon-like peptide-1, glucagon-like peptide-2, oxyntomodulin, and glicentin, and is shown to be expressed primarily in pancreatic α-cells, and L-cells of the ileum and large intestine. It is well established that the control of GCG transcription is under nutrient and hormonal control and can exhibit cell-type specific regulation. Studies using animal models have previously implicated expression differences in both α- and L-cells types following bariatric surgery². In general, cfmRNA levels are dependent on both the expression levels and the apoptotic rate of the cells releasing the nucleic acids into the circulatory system. The captured correlation and temporal trends in GCG cfmRNA levels in this assay could be suggestive of either transcriptomic changes, reduced rate of pancreatic cell death or both.

Numerous high throughput studies in the art have demonstrated the potential of cfmRNA as biomarkers, especially in the field of cancer diagnostics in the form of liquid biopsies. However, few have explored the utility of tissue-specific cfmRNA as biomarkers for metabolic outcomes. In this study, the inventor of the present disclosure demonstrated that a multiplex RT-qPCR approach can be used for the amplification and quantification of tissue-specific cfmRNA, especially those of pancreatic specificity.

Tissue-specific cfmRNAs comprise genes that are expressed at higher levels in the specified subset of tissues relative to the baseline expression across all tissues. These tissue-specific genes often play critical roles in maintaining biological functions unique to those tissues. Recent studies in the art have also established that changes in tissue-specific cfmRNA levels correlates to progression in diseases such as Alzheimer's disease, where a significant portion of dysregulated cfmRNA are found to be brain-specific.

Within the metabolic context of bariatric surgery, the inventors of the present disclosure postulate that post-surgical improvements in glucose homeostasis after surgery can be captured by characterizing pancreatic-specific cfmRNA. By focusing on the subset of pancreatic-specific genes identified by the Human Protein Atlas, the multiplex RT-qPCR approach was able to simultaneously amplify and quantify 13 out of 14 intended pancreatic specific cfmRNA from individual plasma samples. As the plasma-based cfmRNA assay is non-invasive in nature, the inventors of the present disclosure were able to obtain multiple measurements at pre-surgery and post-surgery. Temporal analysis of Ct values obtained from screening pancreatic-specific cfmRNA led to the identification of a small but consistent decrease in levels of circulating GCG cfmRNA during short-term post-surgery.

In addition, the inventors of the present disclosure observed significant correlations of GCG cfmRNA to other clinical measurements (% EWL and HbA1c) obtained across sampled time points. The observed trends suggest that metabolic changes in pancreas can be captured via the plasma, although further studies will be needed to confirm the exact source of GCG cfmRNA in the plasma which could originate from α or L cells. Nevertheless, the demonstrated ability to survey multiple gene targets of pancreatic specificity indicate that such a multiplex RT-qPCR approach can be used as a proxy for non-invasive molecular evaluation of tissue specific molecular alterations in patients with metabolic diseases.

The data of the present disclosure supports the notion that small amounts of pancreatic-specific cfmRNA can be amplified from the plasma and be used as a tool to identify transcriptional alterations of the pancreas that might otherwise be difficult to access and monitor. Furthermore, the protocol of the present disclosure highlights an alternative to whole cfmRNA transcriptome sequencing approaches. By focusing only on pancreatic specific cfmRNA, the approach of the present disclosure allows for much affordable measurements via qPCR, and subsequently reduce the cost of deployment for scalable applications such as patient stratification.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

FIG. 1 shows a schematic diagram of the workflow of reverse transcription and amplification of a nucleic acid (such as cfmRNA) as described in the experimental section. A nucleic acid (such as cfmRNA), spiked-in with luciferase RNA control, was reverse transcribed with Superscript™ III Reverse Transcriptase (Invitrogen, Cat no. 18080044). The product was amplified with rhPCR primers and Platinum™ Taq DNA Polymerase (Invitrogen, Cat no. 10966) using emulsion and CoT PCR. The residual primers were removed with Exonuclease I (New England Biolabs, Cat no. M0293). Amplified products were used for qPCR quantification and sequencing.

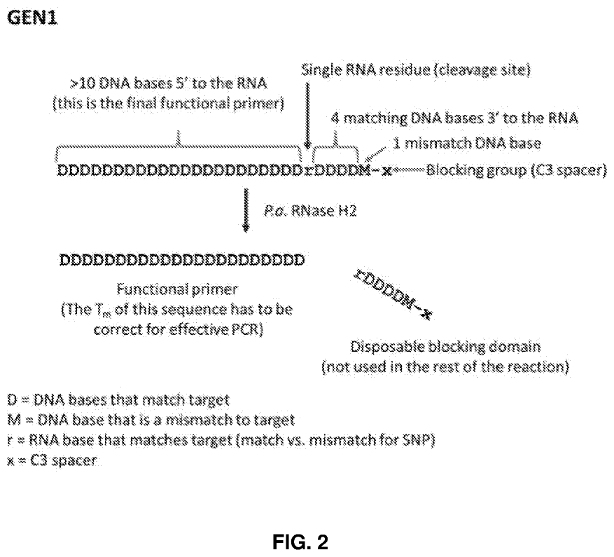

FIG. 2 shows the design of a rhPCR primer as used in the present disclosure. In one embodiment, the rhPCR primer may consists of five different parts: starting from the 5′ end with a functional primer, the cleavage site, four matching DNA bases, one mismatch DNA base, and a blocking group at the 3′ end. Cleavage of the RNA residue by RNase H2 will take place when the primer is perfectly complementary to the template, releasing the 3′ blocking group.

FIG. 3 shows Ct curves of housekeeping genes validating the protocol in amplification of housekeep genes found in plasma. Each curve is a biological replicate. Addition of CoT amplification decreases the CT cycle and improves sensitivity.

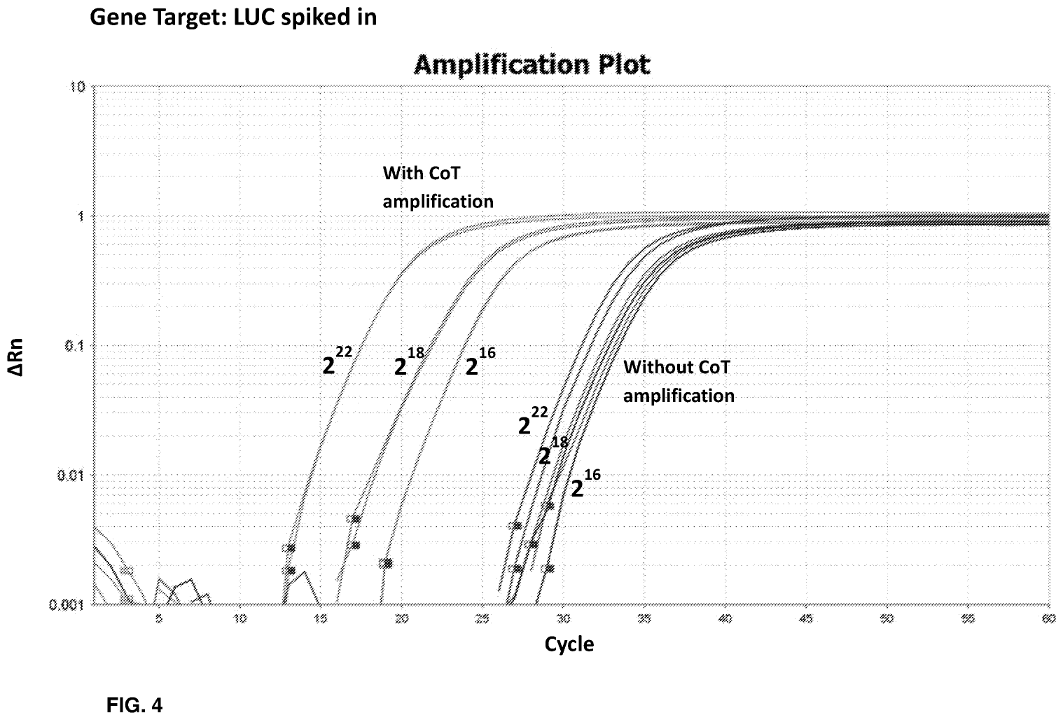

FIG. 4 shows Ct curves of LUC spiked-ins across a range of spiked in molecules. Each curve illustrates a different LUC spiked in. Addition of CoT amplification decreases the CT cycle and improves sensitivity.

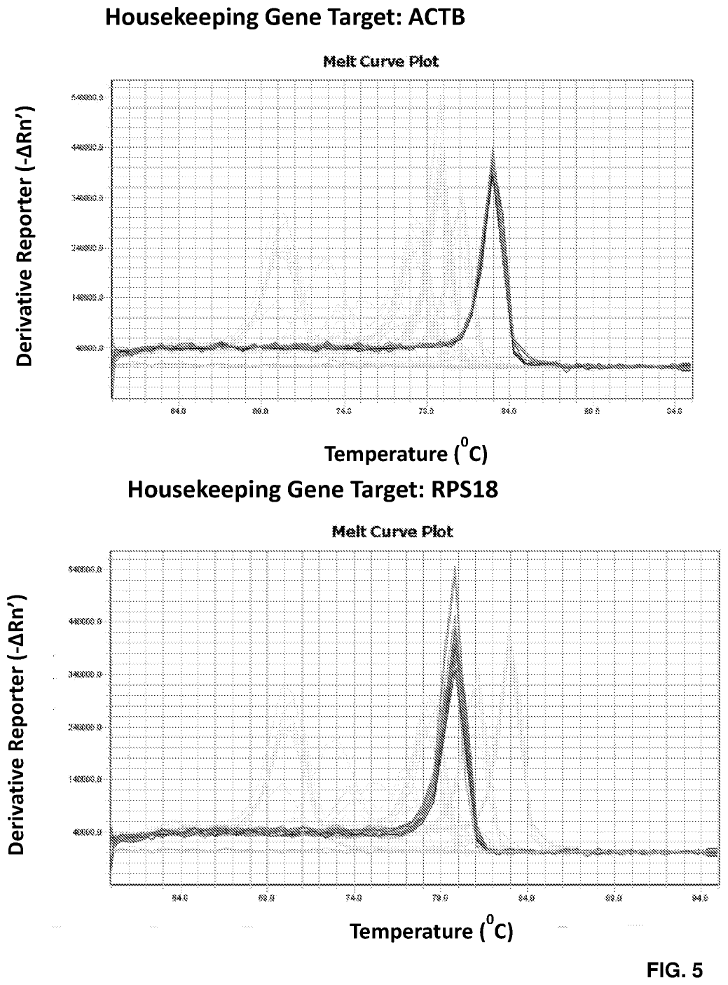

FIG. 5 shows melt curves of housekeeping genes validating the protocol in amplification of housekeep genes found in plasma. Each curve is a biological replicate. Sharp peaks indicating strong and specific amplification of the target at the intended melting temperature.

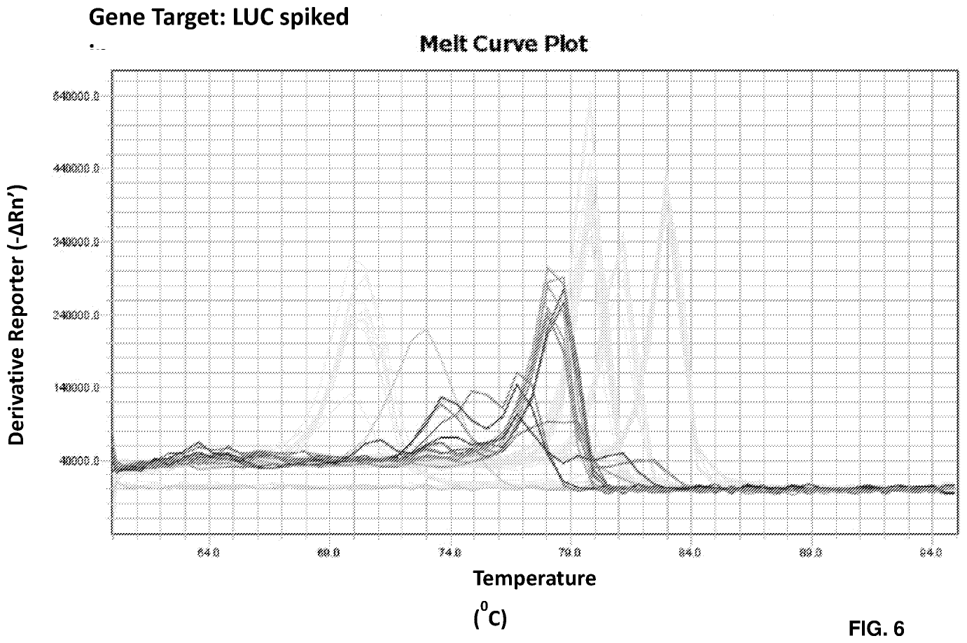

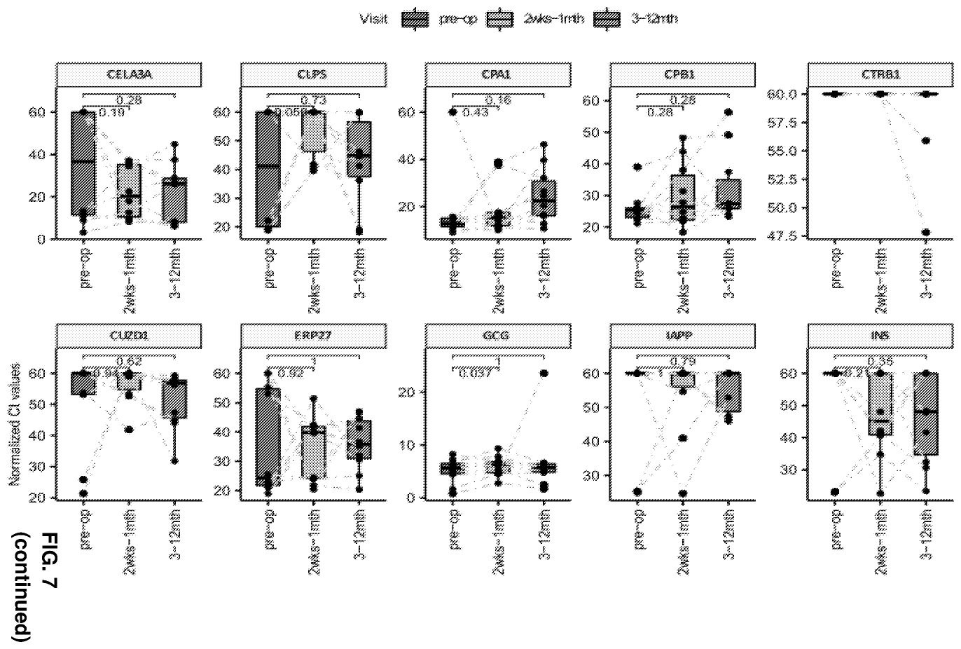

FIG. 6 shows melt curves of LUC spike-in targets validating the protocol in amplification of housekeep genes found in plasma. Each curve is a biological replicate. Sharp peaks indicating strong and specific amplification of the target at the intended melting temperature.

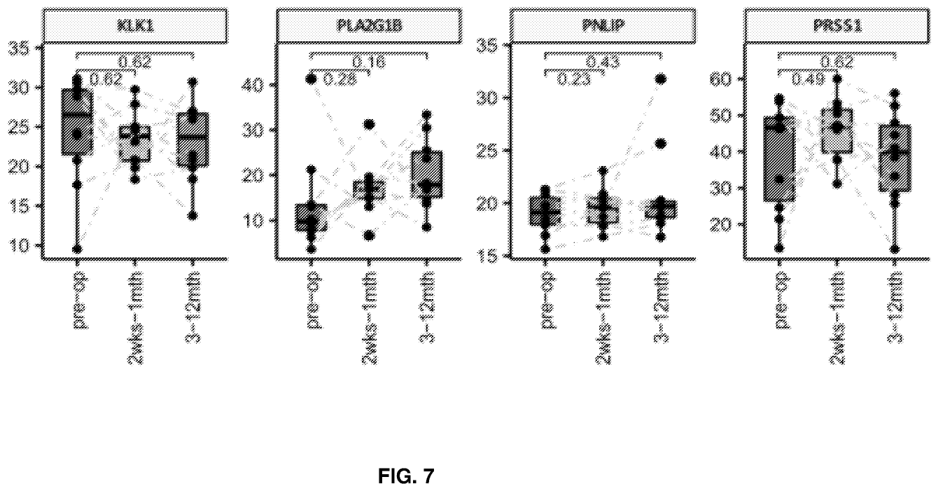

FIG. 7 shows bar charts comparing the levels of pancreatic-specific transcripts across different visits post-surgery. The X-axis represents the different time points and the Y-axis is the normalized Ct values of the cfmRNA. Wilcox paired test is used to statistically compare the short-term and long-term cfmRNA levels against the pre-surgery measurements. GCG cfmRNA Ct value measurements were identified to be significantly higher statistically in short-term post-surgery which translates to lower quantities of GCG cfmRNA in circulation immediately after bariatric surgery.

FIG. 8 shows plots of levels of GCG cfmRNA correlating with clinical biomarkers of HbA1c and % EWL (% of excess weight loss). Ct value measurements of GCG cfmRNA across all patients and visits were correlated with % EWL and HbA1c levels. Linear regression captures a significant positive correlation with % EWL and an inverse correlation with HbA1c.

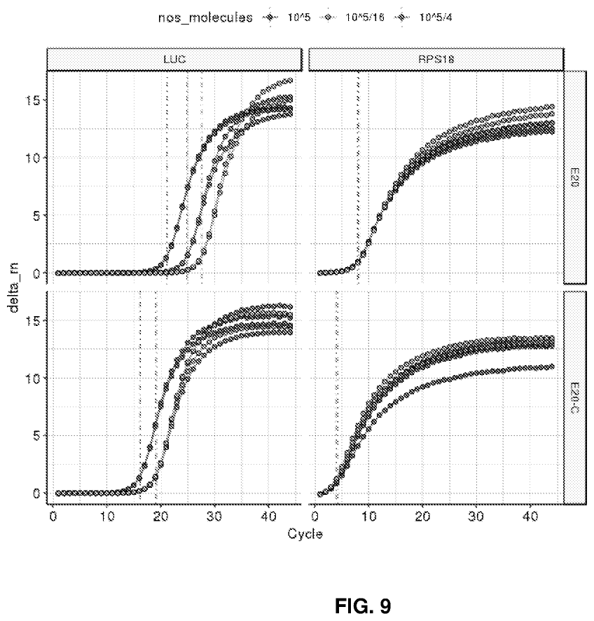

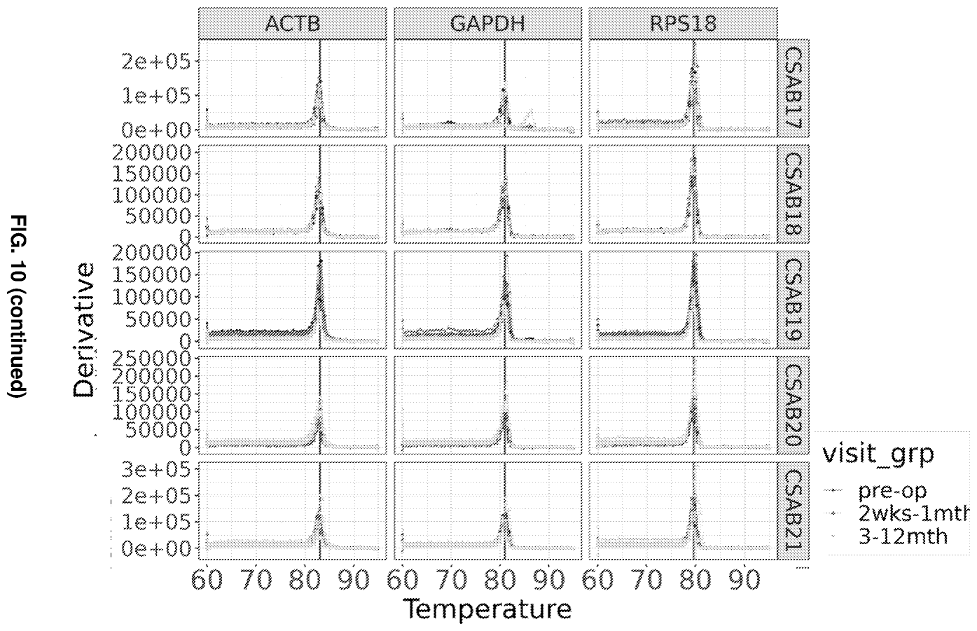

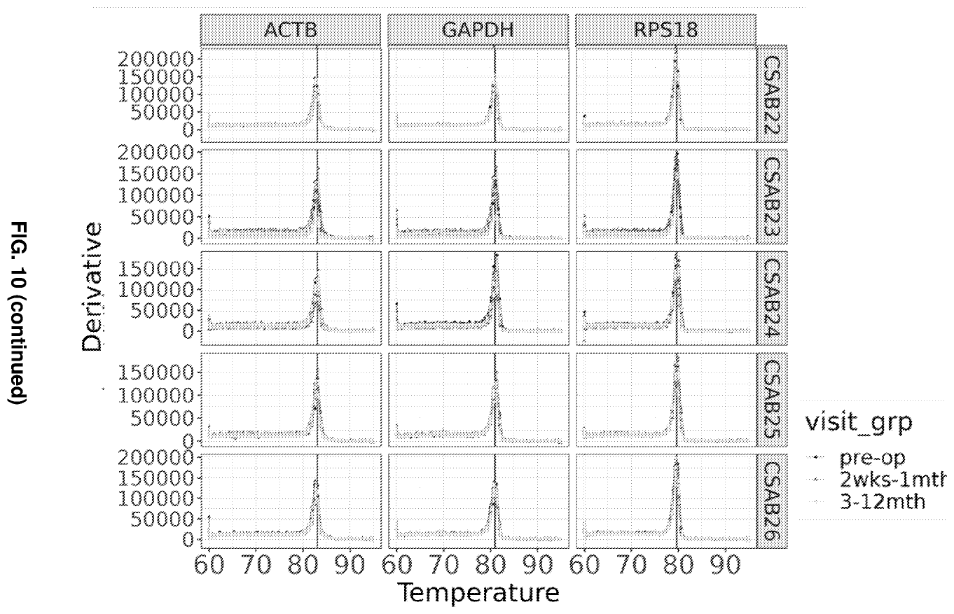

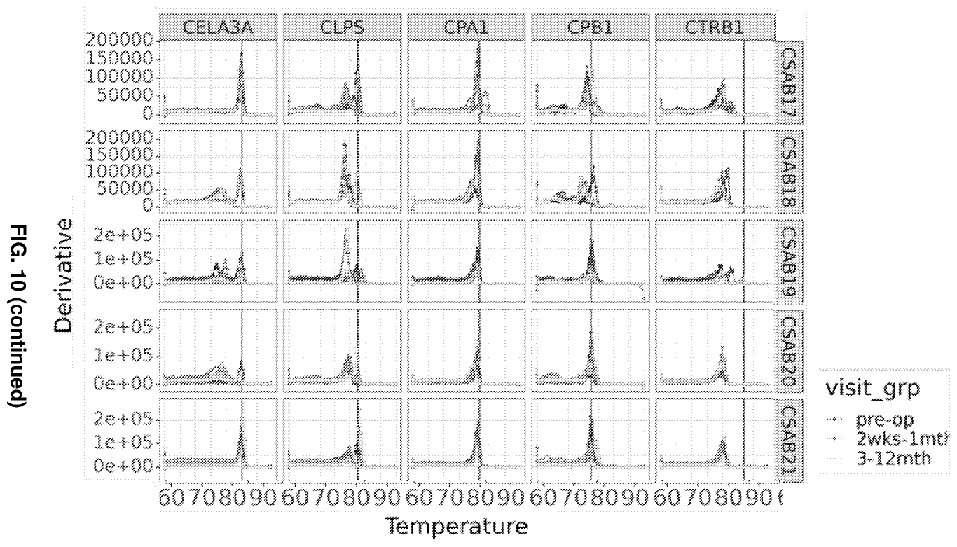

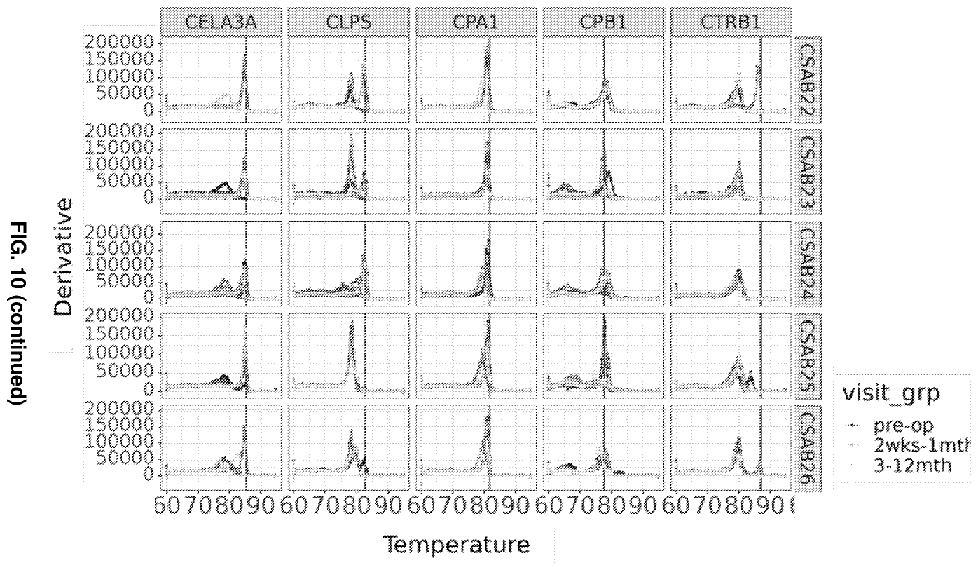

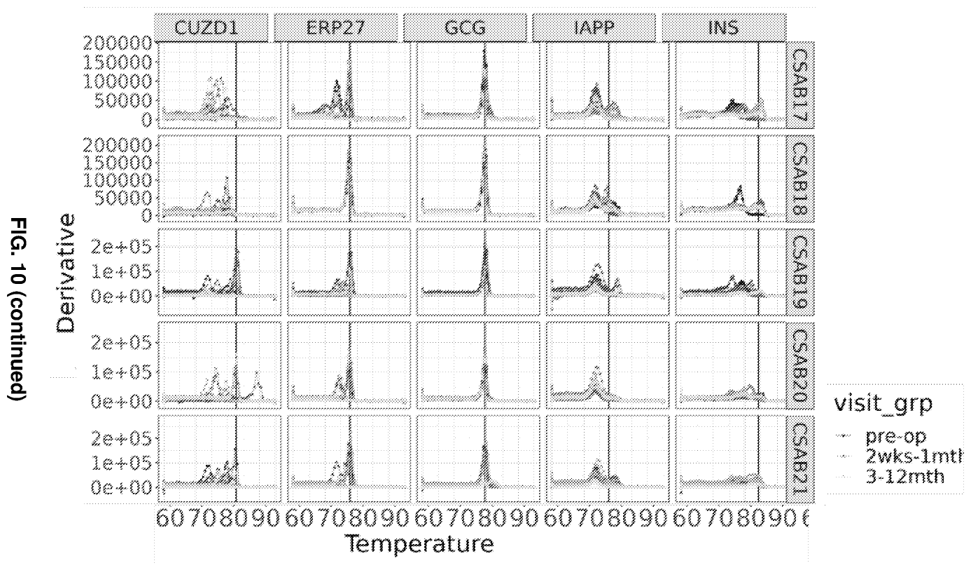

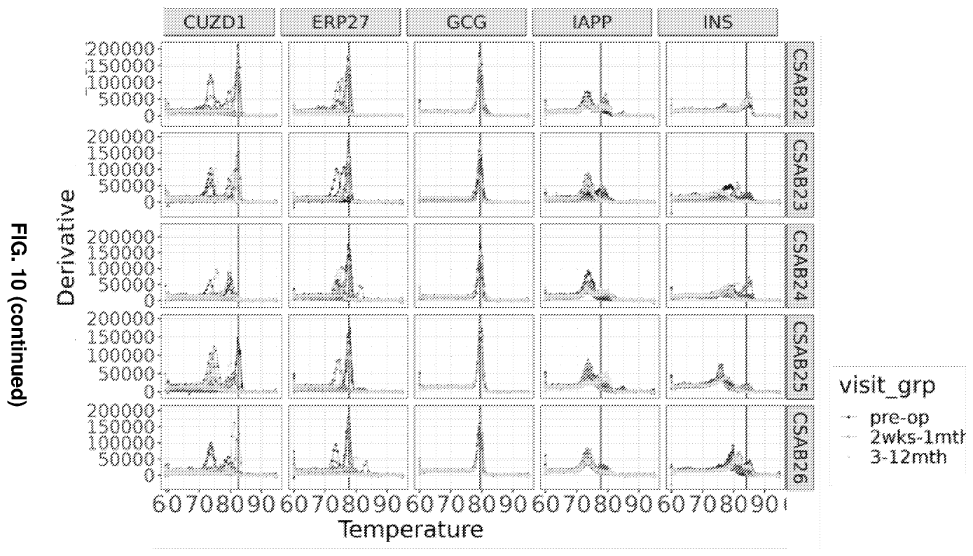

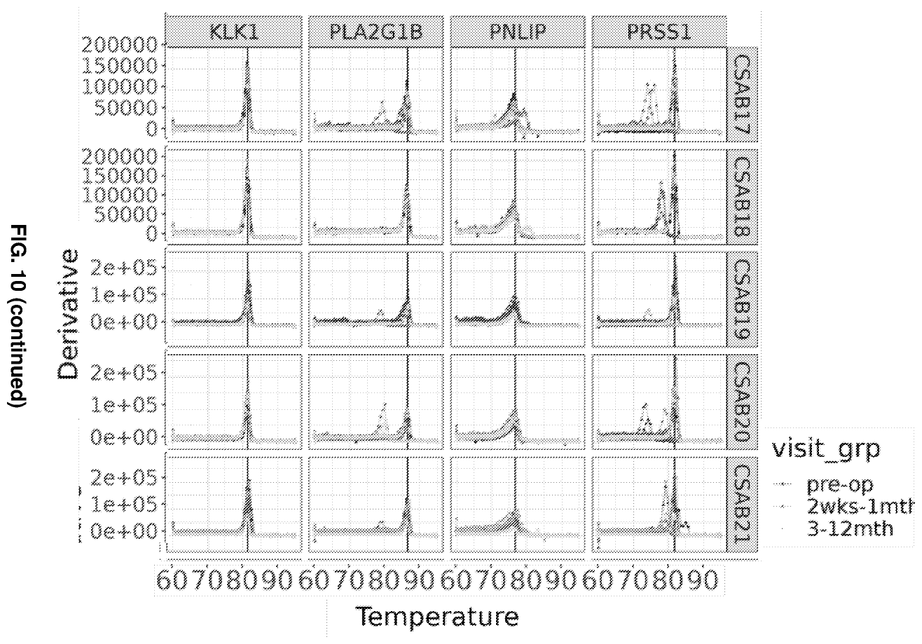

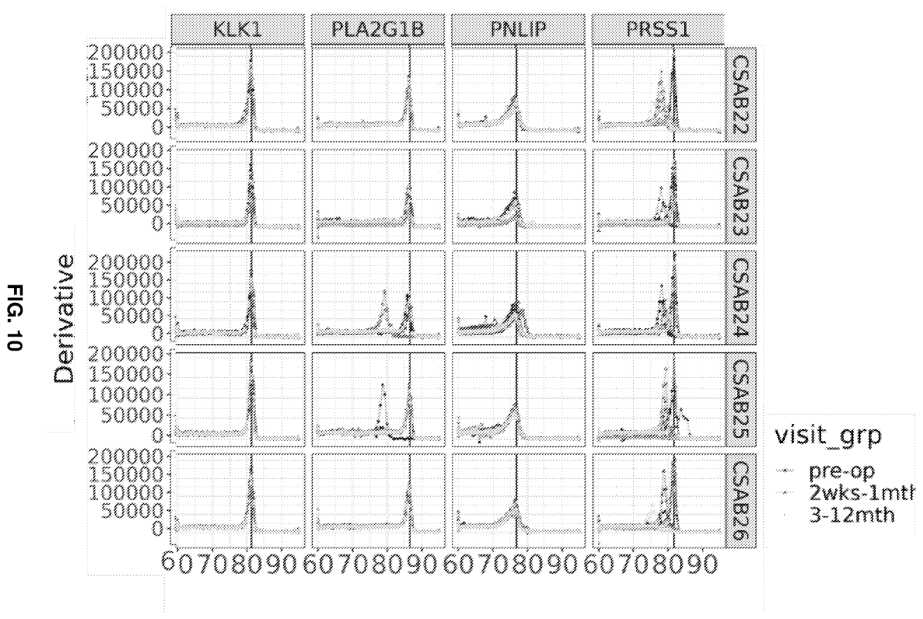

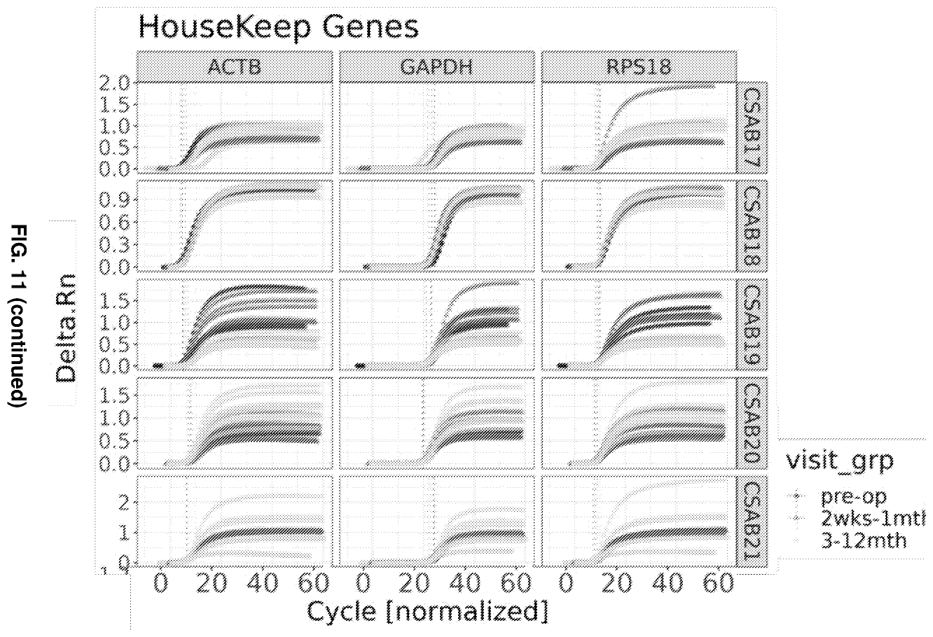

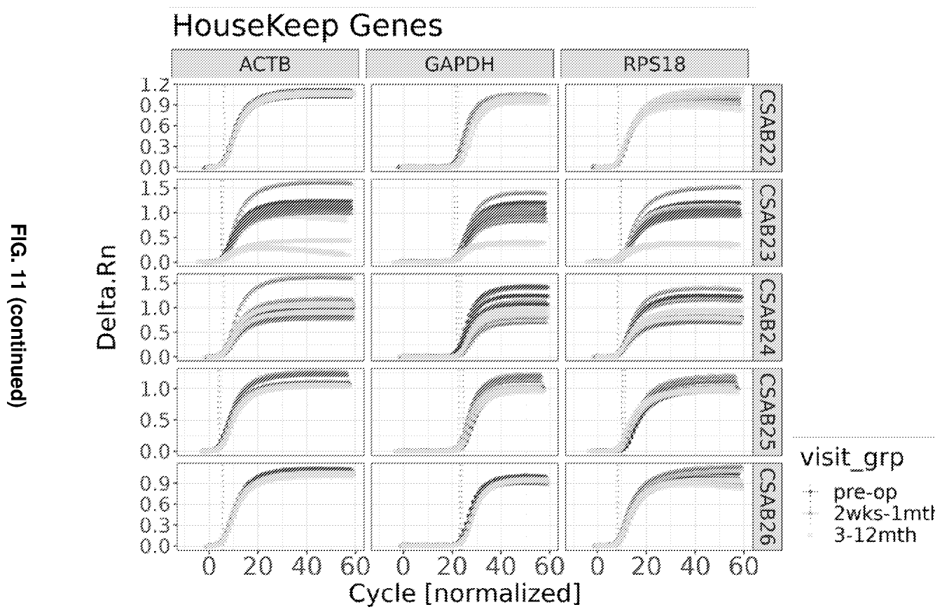

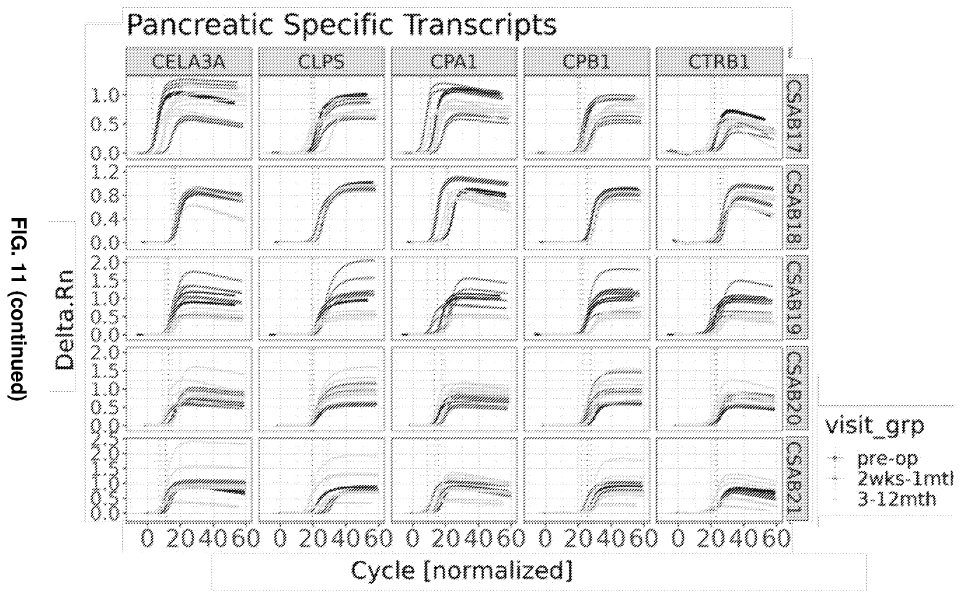

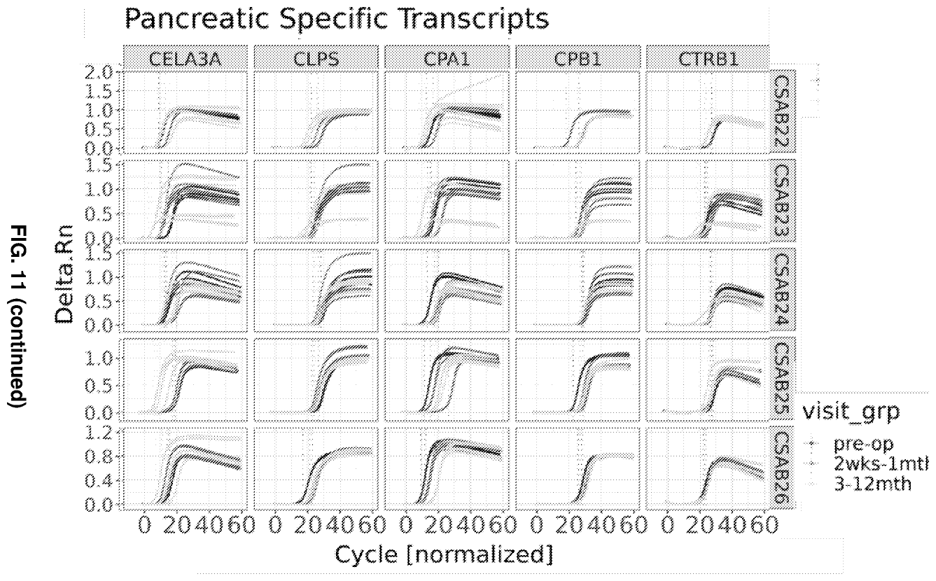

FIG. 9 shows qPCR curves for illustration of the synergistic effects of emulsion PCR, Cot PCR with rhPCR. Each column is a target. There are two targets: RPS18 housekeeping gene and Luciferase (LUC) where LUC is spiked in at different input number of molecules [10⁵ molecules, 1/4*10⁵ molecules, 1/16*10⁵ molecules]. Top row is performed using emulsion rhPCR. Bottom row is performed using emulsion rhPCR and CoT. The qPCR curves are left shifted in the bottom row indicating that CoT decreases the number of cycles required for detection, hence higher sensitivity.

FIG. 10 shows graphs of melting curves of the examined pancreatic genes across the patient cohort at different time points.

FIG. 11 shows graphs of amplification curves the examined pancreatic genes across the patient cohort at different time points.

### APPLICATIONS

Embodiments of the methods disclosed herein provide a fast and efficient way of amplifying a target nucleic acid that can be found in a sample only in small amounts. Embodiments of the disclosed methods also seek to overcome the problems of providing a method of amplifying a target nucleic acid with increased sensitivity.

Advantageously, the methods and/or mixtures as disclosed herein provides a one pot amplification of low input nucleic acid (such as RNA) using a combination/multiplex emulsion-based PCR amplification using specific primer/probe design (such as rhPCR primer design).

Even more advantageously, the methods and/or mixtures as disclosed herein provides a robust non-chemical-based method of recovering emulsion PCR product using freeze-thaw cycle.

The present disclosure also advantageously provides for the inclusion of quality control method of normalizing PCR efficiency that utilizes spiked in luciferase RNA.

The present disclosure also provides an amplification (such as PCR) cycling protocol that leverages the CoT effect for increased sensitivity with minimal loss in linearity using in quantitation.

The present disclosure also provides an adaptable end point where amplified cDNA exhibits compatibility for downstream quantification using either qPCR or next-generation sequencing,

The present disclosure also provides a sensitive and multiplex method for targeted amplification and quantification of pancreatic specific transcripts for monitoring pancreatic health.

The present disclosure also provides a high sensitivity multiplex quantification of circulating pancreatic specific transcripts in plasma for monitoring pancreatic health during bariatric surgery.

The present disclosure also provides the design, evaluation and screening of pancreatic-specific primers that captures temporal changes during the process of bariatric surgery.

The present disclosure also quantifies pancreatic health in other pancreatic driven disease states.

The present disclosure also discloses methods that improve the sensitivity and specificity of an assay for detecting cfmRNA from the plasma (such as COT PCR in conjunction with rhPrimers to achieve better sensitivity).

The present disclosure also involves a combination and interface of multiple techniques that include rhPCR, emulsion as well as COT PCR.

The present disclosure also involves integrating of CoT concept at the qPCR stage for direct quantification, rather than use of CoT for enrichment by depletion of background followed by PCR.

The present disclosure also involves the development of an entire workflow that interfaces each step into a single protocol specifically for cfmRNA, such as interfacing CoT with emulsion and rhPCR.

The present disclosure also discloses pancreatic targets such as GCG for quantifying general metabolic health of the pancreas (i.e., not restricted to prediction of specific diseases such as diabetes). GCG is found to correlate well with clinical indications.

The present disclosure also discloses techniques that work with plasma and embodiments of the methods in addition to the qPCR so as to enable higher sensitivity in detection.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method for assessing/monitoring the health of a subject, the method comprising determining, detecting or quantifying a pancreas-associated polynucleotide in a sample from the subject.

2. The method according to claim 1, wherein the pancreas-associated polynucleotide comprises a pancreas-associated RNA, optionally the RNA comprises a cell-free and/or circulating RNA.

3. The method according to claim 1, wherein the RNA comprises a messenger RNA (mRNA) or part thereof.

4. The method according to claim 1, wherein the method is performed before, during and/or after the subject is being administered a treatment regimen, optionally the method is performed pre and/or post-bariatric surgery.

5. The method according to claim 1, the method comprising:
annealing the target nucleic acid in the presence of a control nucleic acid, and
subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

6. The method according to claim 1, wherein the amplification step of the nucleic acid is performed in the presence of three parts surfactant to one part amplification mixture, optionally the method comprises two amplification steps.

7. The method according to claim 1, wherein the method further comprises a step of freeze and thawing the amplified mixture.

8. The method according to claim 1, wherein the method further comprises performing one or more of the following: RNaseH2-dependent PCR, emulsion PCR, and/or CoT PCR.

9. The method according to claim 1, wherein determining, detecting or quantifying the pancreas-associated polynucleotide comprises performing quantitative polymerase chain reaction (qPCR).

10. The method according to claim 1, wherein the method further comprises reverse transcribing the pancreas-associated RNA.

11. The method according to claim 1, wherein the method further comprises subjecting the products obtained from the preceding step (e.g., the cDNA obtained after reverse transcribing the pancreas-associated RNA) to RNaseH2-dependent polymerase chain reaction (rhPCR).

12. The method according to claim 1, wherein the method further comprises subjecting the products obtained from the preceding step to CoT PCR.

13. The method according to claim 1, wherein the method further comprises adding a control nucleic acid to the sample, optionally wherein the control nucleic acid is added to the sample at a constant amount to thereby normalizes of the amplification efficiency across a plurality of samples, optionally the control nucleic acid is added to the sample at about 102 to 1010 copies.

14. The method according to claim 1, wherein the sample comprises a blood sample, optionally the blood sample is a plasma sample.

15. The method according to claim 1, wherein the pancreas-associated polynucleotide comprises the mRNA of a pancreatic-specific gene or part thereof.

16. The method according to claim 1, wherein the pancreatic-specific gene is at least one selected from the group consisting of KLK1, CTRB1, ERP27, IAPP, PRSS1, CELA3A, PLA2G1B, PNLIP, CUZD1, CPB1, CPA1, CLPS, INS and GCG.

17. The method according to claim 1, wherein the pancreatic-specific gene comprises GCG.

18. The method according claim 1, wherein the subject has, is suspected to have, or is diagnosed to have a metabolic disease.

19. A primer or a primer set comprising one or more primers having a sequence listed in Table 1, Table 2, Table 3 and/or Table 4, or a part thereof, or a sequence sharing at least about 70% sequence identity thereto.

20. A kit for assessing/monitoring the health of a subject, the kit comprising one or more of the following:
one or more primers/primer set primers having a sequence listed in Table 1, Table 2, Table 3 and/or Table 4, or a part thereof, or a sequence sharing at least about 70% sequence identity thereto,
a control polynucleotide;
an rhPCR primer; and
a surfactant.
